(19) United States
(12) Patent Application Publication
(10) Pub. No.: US 2023/0019568 A1
(43) Pub. Date: Jan. 19, 2023

(54) BIOMOLECULE FOR TREATMENT OF CORNEAL PATHOLOGIES

(71) Applicants: Board of Supervisors of Louisiana State University and Agriculture and Mechanical College, Baton Rouge, LA (US); University of Southern California, Los Angeles, CA (US)

(72) Inventors: Haydee BAZAN, New Orleans, LA (US); Nicolas G. BAZAN, New Orleans, LA (US); Thang L. PHAM, New Orleans, LA (US); Bokkyoo JUN, Geismar, LA (US); Nicos A. PETASIS, Los Angeles, CA (US)

(21) Appl. No.: 17/783,770

(22) PCT Filed: Dec. 9, 2020
(86) PCT No.: PCT/US2020/064042

§ 371 Date: Jun. 9, 2022

Related U.S. Application Data
(60) Provisional application No. 62/945,580, filed on Dec. 9, 2019.

(51) Int. Cl. A61K 31/202; A61K 9/00; A61K 9/08; A61P 27/02

(52) U.S. Cl. CPC A61K 31/202; A61K 9/0048; A61K 9/08; A61P 27/02

(57) ABSTRACT

This invention is directed to compositions and methods for treating cornea pathologies. Specifically, aspects of the invention are drawn to a biomolecule and methods of using the same to treat cornea pathologies that affect tissue innervation.

This application claims priority from U.S. Provisional Application No. 62/945,580, filed on Dec. 9, 2019, the entire contents of which is incorporated herein by reference.

### GOVERNMENT INTERESTS

This invention was made with government support under Grant No. R01 EY019465 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to compositions and methods for treating cornea pathologies. Specifically, aspects of the invention are drawn to a biomolecule and methods of using the same to treat cornea pathologies that affect tissue innervation.

### BACKGROUND OF THE INVENTION

Dry eye perturbs vision mainly during aging. It also occurs in rheumatoid arthritis, diabetes, thyroid gland pathologies, environmental conditions (e.g., exposure to smoke or pollutants), long-term use of contact lenses and after refractive surgery. This ocular pathology is triggered by a shortage in tears that lubricate, arrest infections, and nourish and sustain a clear eye surface. Corneal innervation is required to maintain the integrity of the ocular surface, and nerve damage decreases tear production, blinking reflex, and perturbs epithelial wound healing, resulting in loss of transparency and vision.

Axons from sensory nerves from the ophthalmic branch of the trigeminal ganglion (TG) neurons penetrate the corneal stroma surrounding the limbal area and branch out as the subepithelial plexus before reaching the corneal epithelium, finalizing as free nerve endings.

After nerve damage occurs from refractive surgeries, it can take between 3-15 years to recover corneal nerve integrity. As a consequence, corneal sensitivity decreases and dry-eye disease can develop, causing neuropathic pain, corneal ulcers, and in severe cases, the necessity for corneal transplants. In addition, dry eye is linked to cold receptor function, such as the transient receptor potential melastatin 8 (TRPM8) channels that control the corneal surface rate of cooling and maintain normal tear secretion. A decrease in TRPM8 terminals takes place, even long after experimental corneal surgery, indicating that these changes contribute to post-surgery neuropathic pain.

### SUMMARY OF THE INVENTION

The present invention provides methods of protecting the cornea from corneal pathologies.

Further, the invention provides methods of promoting corneal wound healing.

Finally, the invention provides methods of treating a corneal pathology.

In embodiments, the method can comprise administering to the surface of the eye a composition comprising a therapeutically effective amount of RvD6si.

Aspects of the invention provide methods of treating a corneal pathology in a subject in need thereof. For example, the method can comprise administering ocularly to the subject a composition comprising a therapeutically effective amount of:

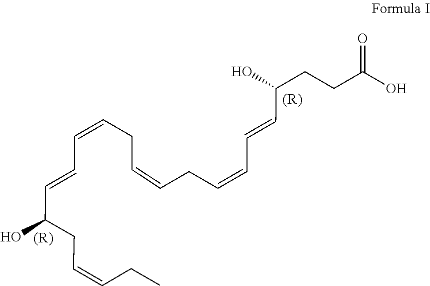

Aspects of the invention further provide methods of protecting the cornea from a corneal pathology in a subject in need thereof. For example, the method can comprise administering ocularly to the subject a composition comprising a therapeutically effective amount of Formula I.

Still further, aspects of the invention can comprise methods of promoting healing of a corneal pathology in a subject in need thereof. For example, methods can comprise administering ocularly to the subject a composition comprising a therapeutically effective amount of Formula I.

In embodiments, treating a corneal pathology comprises increasing corneal nerve density, restoring corneal nerve density, repairing axon growth, inducing Rictor, inducing TIMP8 gene expression, wound healing, or a combination thereof.

In embodiments, the corneal pathology comprises dry eye-disease (DED), photophobia, nerve damage, neuropathic pain, dry eye-like pain, corneal neurotrophic ulcers, trauma, a corneal wound, or neurotrophic keratitis.

In embodiments, the composition further comprises a pharmaceutically acceptable carrier, excipient, or diluent.

In embodiments, the pharmaceutically acceptable carrier, excipient, or diluent is suitable for topical administration.

In embodiments, the composition is formulated for topical administration.

In embodiments, the pharmaceutical composition is formulated as an eye drop.

In embodiments, the composition is administered hourly, daily, weekly, or monthly.

In embodiments, a therapeutically effective amount comprises an amount between about 10 ng and about 1000 ng.

Other objects and advantages of this invention will become readily apparent from the ensuing description.

### BRIEF DESCRIPTION OF THE FIGURES

FIG. 1 shows the identification of Peak 1 as a RvD6si from mouse tears treated with PEDF+DHA. (Panel A) Experimental design with timeframe for injury, treatment, and tear samples collected from 16 corneas. (Panel B) The total ion current (TIC) analysis of 359 m/z compounds (red) in the sample at the RT from 7 to 9.5 min. There are 3 peaks detected with the m/z of 359 which are regarded as dihydroxy-DHA products. In this study, we focus on the peak with a RT of 8.20 min (Peak 1). The LTB4-d4 internal standard (green) eluted at 8.25 min. (Panel C) Full fragmentation analysis of selected Peak 1 and RvD6 standard. (Panel D) Structural interpretation of Peak 1 with the mass of fragmented products after the collision (the dotted red lines represent the broken bonds). The fragments numbered from 1 to 6 were used for the MRM detection. (Panel E) Co-injection of Peak 1 and RvD6. In this run, Peak 1 eluted at 8.10 min while the RvD6 eluted at 8.37 min (the blue color LTB4-d4 internal standard eluted at 8.15 min). All product ions matched with the same difference of RT. (Panel F) The UV diode array profiles for Peak 1 and RvD6 with maximal absorbance at 238.09 nm.

FIG. 2 shows RvD6si derived from added DHA. (Panel A) Structure of RvD6si-d5 with the mass of fragmented products after the collision (the dotted red lines represent the broken bonds). Five deuterium originated from DHA-d5 shift the m/z of RvD6 from 359 (left column) to 364 (right column). The shifted product ions contain deuterium labeling at C21 and C22 (blue color). For MRM detection, one shifted and one non-shifted-product ions were used (red dotted boxes). (Panel B) The MRM detection for RvD6si derived from DHA-d5 (red dotted box) or regular DHA (green dotted box). The transition MRM detection method is shown on top of each graph. The blue color peak is LTB4-d4 internal standard. The merge window shows that RvD6si, derived from the DHA-d5 or DHA, is eluted at the same RT meaning that they are identical compounds. (Panel C) Full fragmentation analysis for the RvD6si-d5 from B. (Panel D) Quantification of RvD6si at increased DHA concentrations. The free DHA and its derivatives such as 14-HDHA, 17-HDHA, and RvD6si were gradually increased as a function of DHA concentration while free AA, and its derivatives 12-HETE and 15-HETE were not.

FIG. 3 shows isolation of RvD6si. (Panel A) The detection of RvD6si from fractionated elution. Samples from tears and media were collected every 30 sec from 6 to 12 min of elution time using a UPLC system with a C18 column before analyzed the presence of synthesized RvD6si by LC-MS/MS. Fractions 6, 7, and 8 were pooled. (Panel B) Mass spectrometry analysis of the combined factions 6-8 to confirm the isolation of RvD6si. The TIC of 359 m/z shows the unique peak while the MRM for all di-hydroxy-DHA products (359→297 and 359→279) scan confirms that there was no other di-hydroxy-DHA derivatives. Moreover, the MRM scan of mono-hydro-DHA (343→299 and 343→281) or tri-hydroxy-DHA (375→277) shows no peaks demonstrating the purity of isolated RvD6si.

FIG. 4 shows RvD6senhance corneal wound healing and sensitivity. (Panel A) Experimental design of wound healing experiments. (Panel B) Representative images of corneal wounded area stained with methylene blue 20 h after injury. (Panel C) The calculated wound closure after injury and treatment. (Panel D) Experimental diagram of cornea sensitivity and collection of cornea and TG tissues. (Panel E) Distribution of recorded corneal sensitivity in non-injured mouse using non-contact aesthesiometer (N=40 corneas). (Panel F) Corneal sensitivity recorded every 3 days. RvD6si-treated mice have significantly higher sensitivity at day 3, 6, and 9 while PEDF+DHA and RvD6 treated groups showed higher cornea sensation only at day 9. At day 12, there was no difference between the tested compounds and vehicle. The statistical p-value is derived from one-way ANOVA, followed by Tukey's honest significant difference (HSD) multiple pairwise comparisons.

FIG. 5 shows RvD6s enhance corneal nerve regeneration. (Panel A) Whole-mount images of normal corneal nerves stained with anti-PGP9.5, a pan-marker for total corneal nerves and SP, a major neuropeptide in the mouse cornea. The insets, which are marked by a dashed box in the whole-mount images, show the amplified center area of the cornea with double PGP 9.5 and SP staining, and PGP 9.5 and SP alone. (Panels B and C) Representative wholemount images and calculated nerve density of PGP 9.5 (Panel B) and SP (Panel C) positive axons at 12 days after injury and treatment. Data was normalized to the baseline (uninjured corneas in Panel A). The statistical p-value is derived from one-way ANOVA, followed by Tukey's honest significant difference (HSD) multiple pairwise comparisons.

FIG. 6 shows changes in the TG transcriptome after cornea injury and RvD6s treatment. (Panel A) The principle component analysis of TG RNA-sequencing data demonstrates well-clustered transcriptional profiles of the three groups of analyzed samples. (Panel B) The Venn diagram of shared up- and down-regulated genes between RvD6 (pink) and RvD6si (green) with vehicle samples as reference. Inputted genes are significantly different from RvD6s to vehicle group (FDR<0.05). (Panel C) The box plot of two significant increase genes in the axonal growth cone classification (GO-0044295). (Panel D) Changes in genes involved in inflammation and pain. (Panels E and G) Evidence of Rictor gene involvement in the nerve regenerated mechanism of RvD6si. (Panel E) The upstream analysis heatmap of RvD6si_vs_vehicle and RvD6_std_vs_vehicle show significant genes changes. The, RICTOR is marked with black bold arrows. (Panel F) The detail signaling pathways of RICTOR in RvD6si_vs_vehicle comparison is shown in the middle-panel. The blue blunt arrows represent inhibited interaction, the red tip arrows represent activated interaction, and the yellow arrows represent conflicted interaction by the IPA analysis. (Panel G) The box plot of Rictor gene expression. The statistical p-value is derived from one-way ANOVA, followed by Tukey's honest significant difference (HSD) multiple pairwise comparisons.

FIG. 7 shows high purity of isolated RvD6si from biological production. The samples from fractions 6 to 8 were pooled and analyzed using LC-MS/MS with specific MRM windows to detect DHA, EPA, and AA and its derivatives including HETEs, LXA₄, PGD₂, PGE₂, PGF₂ alpha. All MRM windows show trace amounts of targeted compounds indicating that the isolated RvD6 is pure.

FIG. 8 shows the gene ontology of cellular components from Enrichr analysis. There are many groups gene located on specific cellular compartments. Among those groups, axonal growth cone (GO: 0044295) group was targeted.

FIG. 9 shows that there are no effective therapies for dry eye and ocular neuropathic pain.

FIG. 10 shows a new RvD6 stereoisomer (RvD6si, topically applied) restores mouse injured cornea.

FIG. 11 shows a new RvD6 stereoisomer (RvD6si) triggers corneal nerve regeneration.

FIG. 12 shows an RvD6 isomer reduces expression of pain-related genes and increases TRPM8 in the trigeminal ganglia.

FIG. 13 shows corneal structure and innervation. Panel A shows the anatomy of human cornea after hematoxylin and eosin histological stain. All five layers are shown: epithelium, Bowman's layer, stroma, Descemet's layer, and endothelium. Panel B shows whole mount view of complete human corneal epithelial nerve network obtained from the left eye of a 45-year-old male donor. Panel C shows detailed course of epithelial nerve bundles running from the periphery to convergence at the center of the cornea (Panels B and C are reproduced with permission from “Elsevier” Reference 5)

FIG. 14 shows incorporation of DHA into PC and PE after 1 h of DHA topical treatment to corneas of mouse with damaged stromal nerves. Panel A shows mice corneas were injured and topical treated with DHA for 1 hr and then lipids extracted and analyzed by LC-MS/MS (27). Proportion of PC and PE containing oleic acid (18:1) in the sn-1 and DHA in the sn-2 position. PE was more enriched in the DHA than PC. Panel B shows release of DHA and synthesis of the monohydroxy-DHA derivatives after corneal injury and topical treatment with PEDF+DHA for three hours. Corneal lipid profiles were analyzed by mass spectrometry-based lipidomic analysis.

FIG. 15 shows lipid mediators derived from the three most abundant essential fatty acid AA, EPA, and DHA esterified in the sn-2 position of the phospholipids. Depending on the primary catalyzing enzyme, cyclooxygenase-2 (COX-2) and, 5 and 15 lipoxygenases (5-LOX, 15-LOX) there is synthesis of variety of bioactive lipids involved in inflammation as well as in resolution of the inflammatory response. Mediators from AA are highlighted in orange, EPA in green and DHA in blue.

FIG. 16 shows structure of an RvD6i. The new isomer was synthesized after topical stimulation of the mouse injured corneas with PEDF+DHA and released in tears. It was analyzed by LC-MS/MS and showed at least 6 matched daughter ions with an RvD6 standard but with an earlier retention time (40). Posterior studies show that the peak retention time coincides with chemically synthesized R,R-RvD6i in a chiral column.

FIG. 17 shows RvD6i accelerate corneal wound healing and sensitivity. Panel A shows representative images of mouse cornea wounded area stained with methylene blue after 20 hours of an injury that damage the epithelial and anterior stroma nerves. The animals received eye drops containing PEDF+DHA or RvD6i in similar concentrations three times per day. The images were taken with a dissecting microscope and quantified using Photoship software (40). Panel B shows recovery of cornea sensitivity at 3, 6, and 9 days after injury and treatment with PEDF+DHA or RvD6i (3×/day) using a non-contact aesthesiometer. RvD6i treated mice recover sensitivity sooner than PEDF+DHA treated corneas. Panel C shows expression of genes involved in inflammation and pain in the TG of RvD6i and analyzed by RNA sequencing (40). Calcb and Tac1 genes were down-regulated while (Panel D) Trpm8 and Rictor genes were up-regulated in the TG neurons by cornea treatment with RvD6i.

FIG. 18 shows schematic model of signaling stimulated by the combination of PEDF+DHA. DHA is rapidly incorporated in membrane phospholipids from corneal epithelium and then released after stimulation by PEDF of the PEDF-R with iPLA2ζ activity. Free DHA is then the substrate for docosanoids such as NPD1 and the novel RvD6i. These docosanoids are then released into tears and by autocrine stimulation to an undefined GPRC receptor(s) that induces the gene and protein expression of neurotrophic factors NGF, BDNF, and Sema7A that are secreted into tears and enhance axon outgrowth. RvD6i stimulates corneal wound healing, corneal sensation and nerve recovery, and tear secretion. The mechanism involves changes in the TG transcriptome with activation of genes related to neurogenesis and modulation of genes implicated in neuropathic pain. Treatment with PEDF or DHA alone does not activate these pathways, and therefore, there was no increase in cornea nerve regeneration (19).

### DETAILED DESCRIPTION OF THE INVENTION

Described herein is the discovery of a stereospecific Resolvin D6-isomer (RvD6si) released in tears that is activated by the neurotrophin pigment epithelium-derived factor (PEDF) plus docosahexaenoic acid (DHA) upon corneal injury. The new RvD6si promotes corneal wound healing, sensitivity, nerve regeneration, and functional recovery by restoring the high-density innervation that sustains ocular surface integrity. After sensing corneal nerve injury and being treated with RvD6si, the transcriptome of the trigeminal ganglion (TG) enhances the gene expression of Rictor, the rapamycin-insensitive complex-2 of mTOR (mTORC2), as well as the expression of genes involved in axon growth, whereas genes related to neuropathic pain are decreased. The new RvD6 isomer stimulated signaling back to the trigeminal ganglia neurons. The new RvD6 isomer induces a genetic program in the trigeminal ganglia that repairs axon growth and decreases neuropathic pain. As a result, attenuation of ocular neuropathic pain and dry eye takes place. Thus, RvD6si opens up new therapeutic avenues for corneal pathologies, such as those that affect tissue innervation, including, but not limited to, neurotrophic keratitis and dry eye-like pain.

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the advantageous methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the disclosure will employ, unless otherwise indicated, techniques of medicine, organic chemistry, biochemistry, molecular biology, pharmacology, toxicology, and the like, which are within the skill of the art. Such techniques are explained fully in the literature.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn to methods of protecting the cornea of a subject. For example, in an embodiment, the method comprises administering to the surface of the eye of a subject a composition comprising a therapeutically effective amount of RvD6si.

Compound

An embodiment of a biomolecule described herein has the following structure of Formula I:

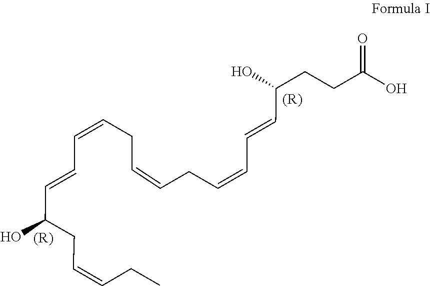

Formula I refer to (4R,5E,7Z,10Z,13Z,15E,17R,19Z)-4,17-dihydroxydocosa-5,7,10,13,15,19-hexaenoic acid. In embodiments, the terms Resolvin D6 stereospecific isomer (RvD6si), RvD6 isomer, RvD6s, RvD6i, or stereospecific Resolvin D6-isomer, 4R,17R-dihydroxy-DHA, can be used interchangeably, and can refer to biomolecules such as Formula I. However, it is to be understood that such terms are not necessarily limited only to a biomolecule according to Formula I. In embodiments, for example, the term “RvD6 isomer” or “RvD6 stereospecific isomer” can refer to other isomers of Resolvin D6 besides that of Formula I.

As used herein, the term “isomer” can refer to different compounds that have the same molecular formula. As used herein, the term “stereoisomer” can refer to isomers that have their atoms bonded in the same order but differ in the arrangement of atoms in space. Stereoisomers can refer to “enantiomers” or “diastereomer”. As used herein, the term “enantiomer” can refer to stereoisomers that are non-superimposable mirror images of each other. As used herein, the term “diastereomer” can refer to stereoisomers that are not mirror images of each other. As used herein, the term “stereospecific” can refer to the conversion in a chemical or enzymatic reaction of one stereoisomer over another.

The cornea is the clear outer layer at the front of the eye. The cornea helps a subject's eye focus light so that the subject can see clearly.

Aspects of the invention can protect against (i.e., prevent) or treat corneal disease or corneal injury, and damage therefrom. “Corneal disease” can refer to any disease or damage of the cornea, such as by various factors, for example, keratitis caused by physical/chemical damage, stimulation, allergy, bacterial/fungal/viral infection, corneal ulcer. It can also refer to corneal epithelial injury (e.g., detachment, corneal erosion), corneal epithelial edema, corneal burn, corneal corrosion due to chemicals, dry eye, and the like. “Corneal injuries” can refer to abrasions (scratches) on the cornea. In certain instances, small injuries can heal on their own; however, deeper scratches or other injuries can cause corneal scarring and vision problems. “Corneal damage” can refer to any damage to the cornea, such as damage caused by, e.g., pathogens, inflammation, physical irritation (e.g., contact lens or UV), chemical irritation (e.g., drug), nerve damage, accumulated fatigue, although not being limited thereto. It can be accompanied by such symptoms as pain, red eye, corneal opacity, dazzling, foreign body sensation, etc. As used herein, the terms “disease”, “injury”, and “dysfunction” can be used interchangeably with “pathology”.

Aspects of the invention can protect against (i.e., prevent) or treat corneal pathologies.

Other aspects of the invention can promote healing of a corneal pathology. The term “promoting healing” or “accelerating healing” can refer to causing a favorable result compared to no treatment. The favorable result comprises, for example, reduction of scarring, reduction of inflammation, regrowth of normal tissue or growth of scar tissue, nerve regrowth, innervation, closure of wound, reduction in infection, and reduction in mortality/morbidity associated with the underlying pathology. Examples of a corneal pathology include, but are not limited to, dry eye-disease (DED), photophobia, neuropathic pain, dry eye-like pain, corneal neurotrophic ulcers, trauma, a corneal wound, neurotrophic keratitis, or a combination thereof. As used herein, the term “neuropathic pain” can refer to pain due to damage to peripheral and/or central sensory pathways or dysfunction of peripheral and/or central sensory pathways, as well as dysfunction of the nervous system.

Allergies, such as to pollen, can irritate the eyes and cause allergic conjunctivitis (which can be referred to as pink eye). This can make one's eyes red, itchy, and watery.

Keratitis refers to inflammation (such as redness and swelling) of the cornea. Infections related to contact lenses are the most common cause of keratitis.

Dry eye occurs when a subject's eyes don't make enough tears to stay wet. This can be uncomfortable and may cause vision problems.

Corneal dystrophies cause cloudy vision when material builds up on the cornea. These diseases usually run in families.

There are also a number of less common diseases that can affect the cornea—including ocular herpes, Stevens-Johnson Syndrome, iridocorneal endothelial syndrome, and pterygium. Aspects of the invention can comprise methods of increasing and/or restoring corneal nerve density, corneal nerve integrity, and/or corneal nerve sensitivity. For example, an embodiment of the invention can comprise a method of treating a corneal pathology in a subject by ocularly administering a composition comprising a therapeutically effective amount of Formula I, wherein treating the corneal pathology comprises increasing corneal nerve density, restoring corneal nerve density, repairing axon growth, inducing Rictor gene expression, wound healing, or a combination thereof. The Rictor gene encodes the RICTOR protein, a key component of the mammalian target of rapamycin-insensitive complex 2 (mTORC2) which plays a role in anti-inflammation and axon growth of sensory neurons after injury. Aspects of the invention can further provide for methods of corneal nerve regeneration and/or innervation. As used herein, the phrase “nerve regeneration” can refer to the repair or regrowth of cells, including neuronal cells. As used herein, the phrase “innervation” can refer to the process of nerves entering a tissue and/or the process of supplying nerves to a tissue, such as a corneal tissue.

Aspects of the invention are also drawn to methods of promoting corneal wound healing. For example, in an embodiment, the method comprises administering ocularly (e.g., to the surface of the eye) to a subject a composition comprising a therapeutically effective amount of Formula I (e.g., RvD6si).

A “wound”, such as a “corneal wound” can refer to physical disruption of the continuity or integrity of tissue structure. “Wound healing” can refer to the restoration of tissue integrity. It will be understood that this can refer to a partial or a fill restoration of tissue integrity. Treatment of a wound thus can refer to the promotion, improvement, progression, acceleration, or otherwise advancement of one or more stages or processes associated with the wound healing process.

Still further, aspects of the invention are drawn towards methods of treating dry eye. The term “dry eye” refers to a multifactorial disease of the tears and ocular surface (including the cornea, conjunctiva, and eye lids) results in symptoms of discomfort, visual disturbance and tear film instability with potential damage to the ocular surface, as defined by the “The Definition and Classification of Dry Eye Disease: Guidelines from the 2007 International Dry Eye Work Shop,” Ocul Surf 2007, 5(2): 75-92). Dry eye can be accompanied by increased osmolarity of the tear film and inflammation of the ocular surface. Dry eye includes dry eye syndrome, keratoconjunctivitis sicca (KCS), dysfunctional tear syndrome, lacrimal keratoconjunctivitis, evaporative tear deficiency, aqueous tear deficiency, and LASIK-induced neurotrophic epitheliopathy (LNE).

The term “subject” or “patient” can refer to any organism to which aspects of the disclosure can be administered, e.g., for experimental, diagnostic, prophylactic, and/or therapeutic purposes. Typical subjects to which compounds of the present disclosure can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

The phrase “pharmaceutically acceptable derivatives” of a compound can include salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs.

“Formulation” as used herein can refer to any collection of components of a compound, mixture, or solution selected to provide optimal properties for a specified end use, including product specifications and/or service conditions. The term formulation can include liquids, semi-liquids, colloidal solutions, dispersions, emulsions, microemulsions, and nanoemulsions, including oil-in-water emulsions and water-in-oil emulsions, pastes, powders, and suspensions. The formulations of the present disclosure can also be included, or packaged, with other non-toxic compounds, such as carriers, excipients, binders and fillers, and the like. The acceptable carriers, excipients, binders, and fillers contemplated for use in the practice of the present invention are those which render the compounds amenable to oral delivery and/or provide stability such that the formulations of the present invention exhibit a commercially acceptable storage shelf life.

The term “administering” can refer to providing a therapeutically effective amount of a formulation or pharmaceutical composition to a subject, using intravitreal, intraocular, ocular, subretinal, intrathecal, intravenous, subcutaneous, transcutaneous, intracutaneous, intracranial, topical and the like administration. The formulation or pharmaceutical compound of the invention can be administered alone, but can also be administered with other compounds, excipients, fillers, binders, carriers or other vehicles selected based upon the chosen route of administration and standard pharmaceutical practice.

In embodiments, the composition is administered “ocularly”, or by “ocular administration”. As used herein, “ocular administration” can refer to topical administration to the eye, without injection. Non-limiting examples of ocular administration include introduction of solution (eye drops), gels, ointments, and colloidal dosage forms (nanoparticles, nanomicelles, liposomes, and microemulsions). Ocular administration is well known in the art (see, e.g., Gaudana et al., 2010, “Ocular Drug Delivery” AAPS J. 12(3): 348-360, incorporated by references herein).

In embodiments, the composition is administered “topically”, or by “topical administration”. The term “topical administration” can refer to application of the composition to a localized area of the body or to the surface of a body part regardless of the location of the effect, such as to the surface of the eye. Typical sites for topical administration include sites on the skin or mucous membranes.

Administration can be by way of carriers or vehicles, such as injectable solutions, topical solutions, or ocular solutions. Suitable solutions include, but are not limited to sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

In embodiments, compositions and formulations will be formulated as solutions, suspensions and other dosage forms for topical administration, such as to the surface of the eye of a subject. Aqueous solutions are can be used, based on ease of formulation, biological compatibility (especially in view of the malady to be treated, e.g., corneal diseases and injuries), as well as a patient's ability to easily administer such compositions by means of instilling one or more drops of the solutions onto the surface of the affected eyes. However, the compositions can also be suspensions, viscous or semi-viscous gels, or other types of solid or semi-solid compositions. Suspensions can be preferred for compositions which are less soluble in water.

As used herein, the term “topical eye drop” can refer to administering a composition to the subject's outer cornea surface as a liquid, gel, or ointment. The term “drop volume” can refer to the amount of an ophthalmically acceptable liquid that resembles a drop. For example, the drop volume can refer to a volume of liquid corresponding to about 5 μL to about 1000 μL, such as about 5 μL to about 500 μL, for example about 5 μL to about 200 μL. In embodiments, the drop volume can comprise about 20 μL.

The formulations or pharmaceutical composition of the present disclosure can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. The pharmaceutically acceptable carriers, excipients, binders, and fillers that can be used in the practice of the disclosure are those which render the compounds of the invention amenable to intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers, silicone, and the like, and can be processed internally by the subject without affecting the effectiveness of the composition/formulation packaged and/or delivered therewith.

Different forms of the formulation can be calibrated in order to adapt both to different individuals and to the different needs of a single individual. In embodiments, the subject can be an individual afflicted one or more corneal pathologies. For example, the subject can be an individual with dry eye syndrome, keratoconjunctivitis sicca (KCS), dysfunctional tear syndrome, lacrimal keratoconjunctivitis, evaporative tear deficiency, aqueous tear deficiency, LASIK-induced neurotrophic epitheliopathy (LNE) ocular herpes, Stevens-Johnson Syndrome, iridocorneal endothelial syndrome, pterygium, damage of the cornea, such as by various factors, for example, keratitis caused by physical/chemical stimulation, allergy, bacterial/fungal/viral infection, corneal ulcer, corneal injuries, dry eye-disease (DED), photophobia, neuropathic pain, dry eye-like pain, corneal neurotrophic ulcers, trauma, a corneal wound, neurotrophic keratitis, or a combination thereof.

The term “therapeutically effective amount” as used herein can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing. As used interchangeably herein, “subject,” “individual,” or “patient,” can refer to a vertebrate, such as a mammal (for example, a human). Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

A therapeutically effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

As used herein, “an ophthalmically effective amount” can refer to an amount of an embodiment of the composition or pharmaceutical composition that, when administered to a patient, prevents, treats or ameliorates corneal disease or corneal injury, or conditions associated thereof. As one example, “an effective amount to treat dry eye” can refer to an amount that, when administered to a patient, prevents, treats or ameliorates a dry eye disease or disorder, or conditions associated thereof.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that is useful in preparing a pharmaceutical composition that is safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” can refer to one and more such excipients, diluents, carriers, and adjuvants.

As used herein, a “pharmaceutical composition” or a “pharmaceutical formulation” can encompass a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. The pharmaceutical composition can be formulated to be compatible with its intended route of administration, such as ocular administration, and effect desired by the practitioner.

In embodiments, the pharmaceutical composition can comprise a therapeutically effective amount of RvD6 isomer and a therapeutically effective amount of one or more additional active agents. Such pharmaceutical compositions (i.e., an RvD6 isomer and an additional active agent) can be referred to as a combination composition. Suitable additional active agents include, but are not limited to one or more anti-oxidants, anti-allergenics, anti-inflammatory agents, anti-viral agents, anti-bacterial agents, pain relievers, moisturizers, lubricants, or antipyretics. For example, the one or more anti-oxidants can be synthetic antioxidants, natural antioxidants, or a combination thereof. In embodiments, the antioxidants can protect the double bonds of RvD6 isomer.

A “pharmaceutical composition” can be sterile, and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, topical, intravenous, buccal, rectal, parenteral, intraperitoneal, intradermal, intratracheal, intramuscular, subcutaneous, by stent-eluting devices, catheters-eluting devices, intravascular balloons, inhalational and the like.

The term “administration” can refer to introducing a composition of the disclosure into a subject. One route of administration of the composition is topical administration. Another route of administration is ocular administration. In embodiments, the composition can be administered to the surface of the eye. However, any route of administration, such as oral, intravenous, subcutaneous, peritoneal, intra-arterial, inhalation, vaginal, rectal, nasal, introduction into the cerebrospinal fluid, intravascular either veins or arteries, or instillation into body compartments can be used.

In embodiments, the composition is administered hourly. For example, the composition is administered continuously, about hourly, about every 2 hours, about every 3 hours, about every 4 hours, about every 5 hours, about every 6 hours, about every 8 hours, about every 10 hours, about every 12 hours, about every 16 hours, about every 18 hours, about every 20 hours, or about every 24 hours.

In embodiments, the composition can be administered daily. For example, the composition can be administered every day, about every 2 days, about every 3 days, about every 5 days, or about every 7 days.

In embodiments, the composition can be administered weekly. For example, the composition can be administered about every week, about every 10 days, about every two weeks, about every 18 days, about every 3 weeks, or about every 25 days

In embodiments, the composition can be administered monthly. For example, the composition can be administered about every month, about every two months, about every 3 months, about every 4 months, about every five months, about every 6 months, about every 7 months, about every 8 months, about every 9 months, about every 10 months, about every 11 months, or about every 12 months. In embodiments, the composition can be adminstered once a year, or more than once a year.

In embodiments, the composition can be administered when symptoms of a corneal pathology first appear, and administration of the composition can cease when symptoms are alleviated or relieved, or a period of time after symptoms are alleviated or relieved.

The frequency of administration can vary depending on the formulation used, the particular condition being treated or prevented, and the patient/subject's medical history. In general, it is preferable to use the minimum dose that is sufficient to provide effective therapy. Patients can be monitored for the effectiveness of treatment using quantitative or test methods suitable for the condition to be treated or prevented, such as corneal pathologies described herein, which is routine to those of ordinary skill in the art.

In embodiments the dosage of the composition administered comprises between about 10 ng and about 1000 ng. For example, the dosage of the composition administered can comprise between about 20 ng and about 500 ng, such as about 50 ng and about 100 ng. In embodiments, the dosage can comprise about 50 ng-about 80 ng.

As used herein, “treatment” and “treating” can refer to the management and care of a subject for the purpose of combating a condition, disease or disorder, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and can include the administration of the active compounds to prevent or reduce the risk of the onset of symptoms or complications.

The patient to be treated can be a mammal, such as a human being. Treatment can encompass any pharmaceutical use of the compositions herein, for example for treating a disease as provided herein.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example A

Docosanoic Signaling Modulates Corneal Nerve Regeneration: Effect on Tear Secretion, Wound Healing, and Neuropathic Pain

The cornea is densely innervated, mainly by sensory nerves of the ophthalmic branch of the trigeminal ganglia (TG). These nerves are important to maintain corneal homeostasis, and nerve damage can lead to a decrease in wound healing, an increase in corneal ulceration and dry eye disease (DED), and neuropathic pain. Pathologies, such as diabetes, aging, viral and bacterial infection, as well as prolonged use of contact lenses and surgeries to correct vision can produce nerve damage. There are no effective therapies to alleviate DED (a multifunctional disease) and several clinical trials using ω-3 supplementation show unclear and sometimes negative results. Using animal models of corneal nerve damage, we show that treating corneas with pigment epithelium-derived factor (PEDF) plus docosahexaenoic acid (DHA) increases nerve regeneration, wound healing, and tear secretion. The mechanism involves the activation of a calcium-independent phospholipase A2 (iPLA2ζ) that releases the incorporated DHA from phospholipids and enhances the synthesis of docosanoids neuroprotectin D1 (NPD1) and a new resolvin stereoisomer RvD6i. NPD1 stimulates the synthesis of brain-derived neurotrophic factor (BDNF), nerve growth factor (NGF), and of semaphorin 7A (Sema7A). RvD6i treatment of injured corneas modulates gene expression in the TG resulting in enhanced neurogenesis; decreased neuropathic pain and increased sensitivity. Taken together, these results validate therapeutic compostions and methods to re-establish the homeostasis of the cornea.

Cornea Anatomy

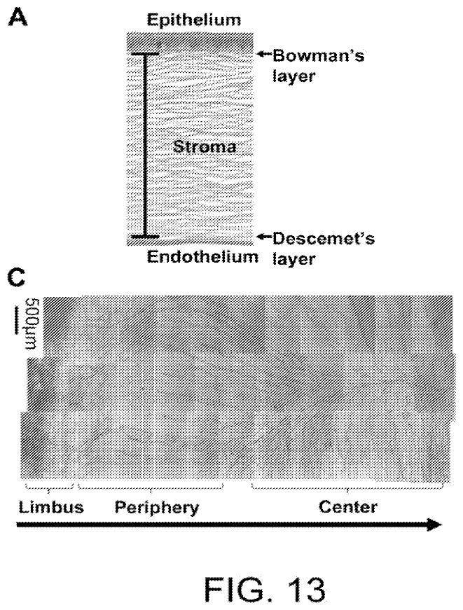

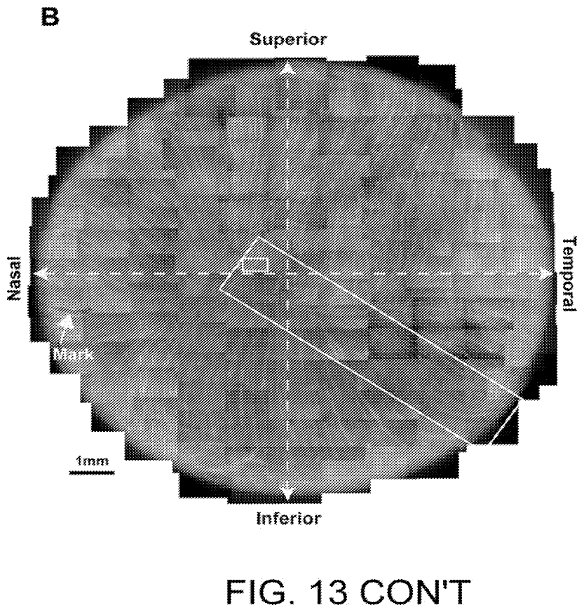

The transparent cornea accounts for 70% of the refractive power of the human eye by allowing light to pass through and be projected to the retina. In addition, the cornea also provides an important barrier to regulate immune response and to prevent pathogens from entering the ocular globe. Anatomically, the cornea can be divided into five sublayers: epithelium, Bowman's layer, stroma or substantia propria, Descemet's membrane, and endothelium (1, 2) (FIG. 13 Panel A).

The epithelium consists of 5-7 layers of nonkeratinized squamous epithelial cells, which are classified into three morphological cell types: superficial epithelial cells, intermediate wing cells, and the innermost basal epithelial cells with high rates of proliferation (2). The epithelial cells are connected by tight junctions that block the passage of foreign materials, such as dust, water, and bacteria, into the eye and provide a smooth surface that absorbs oxygen and cell nutrients. Moreover, the outer-most layer of the epithelium is in contact with the tear film that allows maintenance of the moist of the ocular surface and protects from damage that results from drying (dry eye, DE). Corneal epithelial cells regularly undergo a “turnover” with movement of stem cells from the limbal epithelium to the basal layer. These basal cells move toward the surface to generate two to three layers of wing cells and then begin terminal differentiation and desquamation. On average, the turnover time of human corneal epithelial cells is between 7-10 days (3).

The Bowman's layer is a thin, acellular layer that separates the epithelium from the stroma. It mainly contains collagen IV and laminin. The organization of these proteins is important to maintain the transparency of the tissue.

The stroma layer is built up by quiescent keratocytes and a well-organized extracellular matrix (ECM) composed primarily of highly ordered collagen type 1 fibrils called lamella, and proteoglycans and also constitutes the largest portion of the cornea (about 90% of corneal thickness). The stroma provides structural support to the cornea as well as transparency by facilitating the passage of light through collagen fibrils in a manner that prevents scattering. Keratocytes (the flat cells situated between collagen fibers) are the main cell residents of corneal stroma.

The Descemet's membrane is an acellular thin layer synthetized by the endothelium that is composed of fibronectin, laminin and collagen IV and VII as well as proteoglycans. Damage to the Descemet's membrane produces corneal edema and loss of vision.

The last layer of the cornea is the endothelium, which is in contact with the aqueous humor. It is a monolayer of cells responsible for pumping fluid to regulate corneal stromal dehydration. Without endothelial pumps, there will be stroma edema, which produces opacity and decrease in vision. The human corneal endothelial cells have very low capacity for proliferation, resulting in age-related reduction in cell density.

An important characteristic of the cornea is its dense innervation (FIG. 13 Panel B). Most corneal nerve fibers are sensory in origin and derived mostly from neurons of the ophthalmic branch of the trigeminal ganglia (TG) (4-6). Anatomically, the corneal nerve network originates when stromal nerves enter the corneal sclera limbus in a radial fashion. To maintain corneal transparency, the arriving nerves lose their myelin sheaths and are surrounded by Schwann cells alone. In the stroma, the thick branches divided into smaller nerve branches. Most of the branches penetrate the Bowman's layer in the periphery and run to the center of the epithelium to form the epithelial nerve network (FIG. 13 Panel C) giving life to a dense network of nerve terminals.

Corneal nerves stimulate tear secretion and blinking to maintain the integrity of the ocular surface (7). Alterations in corneal innervation occur in aging, diabetes, immunological diseases, such as rheumatoid arthritis and Sjögren's syndrome, viral and bacterial infection, prolonged use of contact lenses and refractive surgeries, such as laser in situ keratomileusis (LASIK) and photorefractive keratectomy (PRK) (8-13). Complications from nerve damage diminish sensitivity, decrease tear secretion and blinking, and as a consequence, DE disease (DED) that produces neuropathic pain and corneal ulceration in severe cases. Due to the abundance of sensory nerves, the cornea is also a potent generator of pain in the human body.

PEDF+DHA Treatment for Cornea-Related Damage. Discovery of a Resolvin D6 Stereoisomer.

As mentioned, after damage, corneal nerve density slowly and incompletely recovered with decrease in sensitivity and DE symptoms. Studies from our laboratory have shown that application of nerve growth factor (NGF) in conjunction with the ω-3 fatty acid docosahexaenoic acid (DHA) results in faster recovery of corneal nerve density after experimental PRK in rabbits (14). At that time, the mechanisms could be mediated by the DHA-derived lipid mediator neuroprotectin D1 (NPD1), a docosanoid with potent anti-inflammatory and neuroprotective actions (15). Synthesis of NPD1 in retinal pigment epithelial (RPE) cells is stimulated by several growth factors with pigment epithelium-derived factor (PEDF) being 10 times more potent than NGF (16). PEDF is a broad-acting neurotrophic and neuroprotective factor that regulates processes associated with angiogenesis, neuronal cell survival, and cell differentiation (17) and is released from corneal epithelium after injury (18). Later studies have shown that treatment with PEDF+DHA decreases inflammation and stimulates corneal wound healing and nerve regeneration in rabbit and mouse cornea models of experimental surgery, as well as in pathologies like diabetes and herpes simplex virus (HSV1) infection (19-23). The action requires treatment with both, PEDF and DHA (19). A 44-amino acid fragment of PEDF has neuroprotective activity, while an adjacent 34-amino acid peptide has anti-angiogenic activity (24, 25). Comparing the effect of the two peptides with the whole PEDF protein plus DHA in a rabbit model of corneal stroma dissection, we found that, unlike 34-mer-PEDF, 44 mer-PEDF+DHA decreases inflammation and increases tear secretion and corneal sensitivity and also promotes regeneration of corneal nerves by activating a PEDF-receptor (PEDF-R) (21). This transmembrane receptor is expressed in the cornea and has calcium-independent phospholipase A2 (iPLAζ) activity (26, 27) that released DHA, which is enriched in the sn-2 position of membrane phospholipids by DHA supplementation.

Studies on calf corneas identified phosphatidylcholine (PC), phosphatidylethanolamine (PE), and sphingomyelin as the main phospholipids in the tissue (28). Among these phospholipids, PC is the most abundant with the highest content in the epithelium. Similar observations were reported in human (29) and rabbit corneas (30). In the rabbit, oleic acid (18:1) is the dominant fatty acid esterified in phospholipids in all of the corneal layers (about 50% of total fatty acids in phospholipids) followed by palmitic acid (16:0), which comprises about 16-18%. With respect to the polyunsaturated fatty acids (PUFAs) esterified in phospholipids, the higher percentage (about 9% of total fatty acids) corresponds to arachidonic acid (AA), while the percentage of eicosapentaenoic acid (EPA) and DHA esterified in phospholipids is much lower (around 1.6% of total fatty acids) (30).

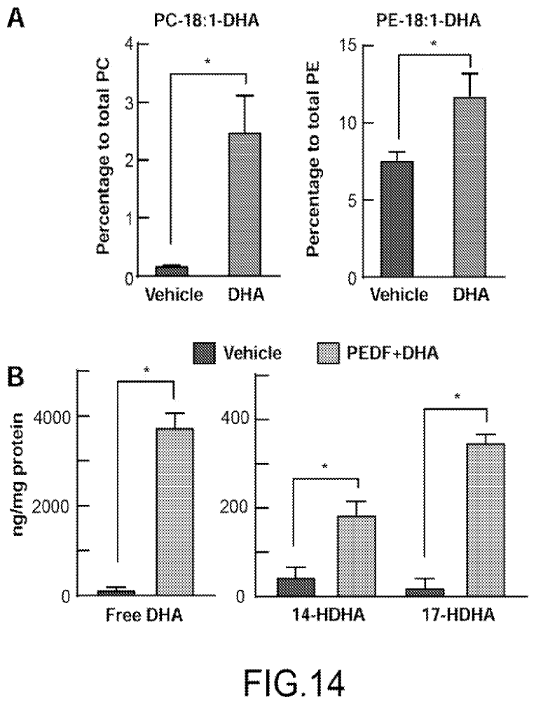

DHA topical treatment of mice corneas, in which stromal nerves had been damaged, produced a rapid incorporation of the fatty acid in PC and PE molecular species containing 18:1-DHA (27), demonstrating that the addition of the PUFAs created a significant enrichment of DHA in the lipid membrane composition (FIG. 14 Panel A).

Tissue damage activates phospholipases A2 that releases PUFAs, such as AA, EPA and DHA, from the sn-2 position (31, 32). Several early studies from our lab and others have demonstrated that the cornea responds to injury, increasing the synthesis of prostaglandins (PGs) by activation of cyclooxygenease-2 (COX-2) (33-36) and hydroxyeicosatetraenoic acids (HETEs) and Lipoxin A4 (LXA4) by activation of lipoxygenases (LOXs) (37-39). Since the concentration of DHA in membrane lipids is very low (FIG. 14 Panel A) and (30), we found that the addition of DHA to the corneas treated with PEDF was important to increasing the synthesis of lipids derivatives of DHA (docosanoids) with strong anti-inflammatory properties (19, 40, 41). Therefore, activating the iPLA2ζ of the PEDF-R by treating the corneas with PEDF+DHA leads to a more than 3000- fold increase of free DHA released from the cornea (FIG. 14 Panel B).

Free DHA is then the substrate for the synthesis of 14- and 17-hydroperoxyDHA (HpDHA) that are rapidly converted in the more stable hydroxy-DHA derivatives (HDHA) (FIG. 14 Panel B). These results confirmed that PEDF+DHA treatment stimulates the formation of docosanoids derived from DHA.

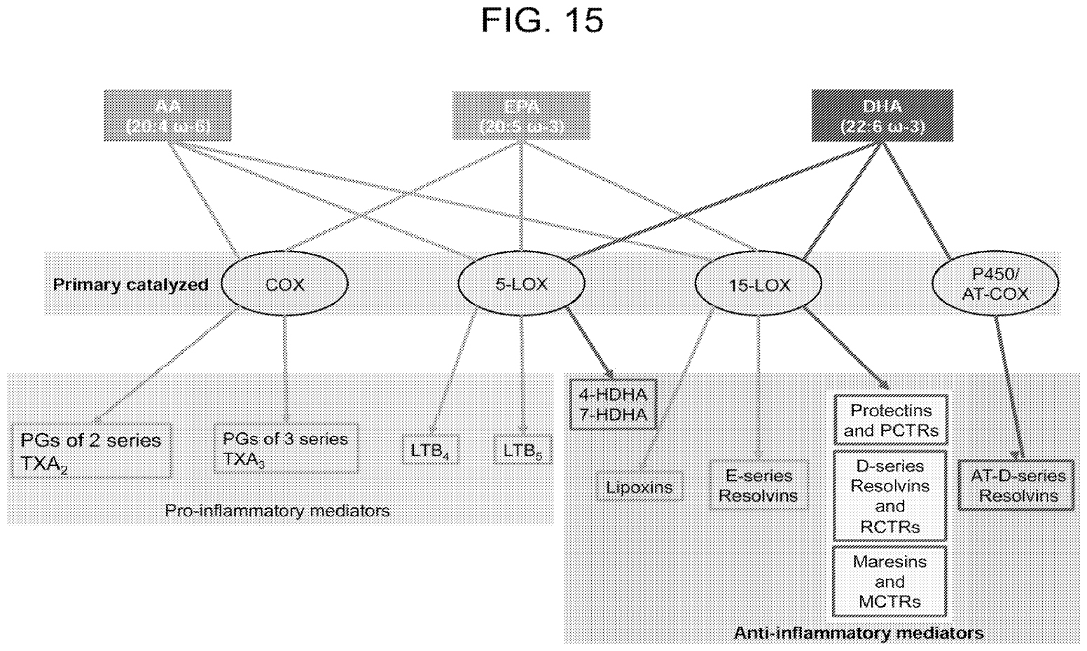

FIG. 15 shows a scheme of bioactive lipids resulting from AA, EPA, and DHA.

While many AA lipid mediators, as well as some EPA lipid mediators, have strong pro-inflammatory properties, all known DHA mediators (the docosanoids) act to protect and resolve inflammation (42, 43). They constitute part of a family named specialized pro-resolvin mediators (SPMs) that includes NPD1 and other protectins, maresins, and resolvins of the D series (43) and the newer sulfide conjugates of protectins (PCTR), maresins (MCTR), and resolvins (RCTR). The synthetic mechanism to produce the SPM involves lipoxygenases (including 15-LOX as primary catalyzer and 5 LOX as secondary catalyzer), cyclooxygenase (in the presence of aspirin), and cytochrome P450 enzymes (44). Information about the signaling mechanisms of DHA lipid mediators is still limited, especially identification of their receptors (Table 1). Most of the known receptors belong to the family of G-protein coupled receptors. In addition, some docosanoids share the same receptor, but their activation exerts specific biological activities (43).

TABLE 1
List of reported receptors of docosanoids.

| | | | Expression |
|---|---|---|---|
| Name | Receptors | References | in the cornea |
| Resolvin D1 | ALX/FPR2, GPR32 | (44) | Yes |
| Resolvin D2 | GPR18 (DRV2) | (45) | ND |
| Resolvin D3 | ALX/FPR2, GPR32 | (46) | Yes |
| Resolvin D4 | N/A | — | — |
| Resolvin D5 | GPR32 | (47) | ND |
| Resolvin D6 | N/A | — | — |
| Neuroprotectin D1 | GPR37 (Pael-R) | (48) | ND |
| Maresin 1 | LGR6 | (49) | ND |
| Maresin 2 | N/A | — | — |

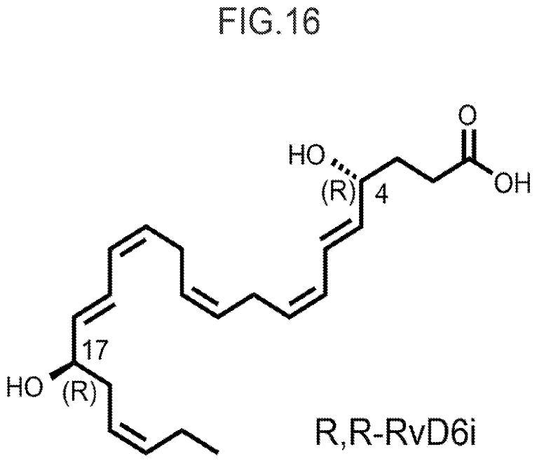

We discovered a new docosanoid, a stereo isomer of resolvin D6 (RvD6), referred to as RvD6i (FIG. 16) that is released in mouse tears after injury and treatment with PEDF+DHA (40). The fragmentation pattern of this new lipid shows at least six matched product ions that coincide with RvD6. Resolvin D6 had been found in some tissues, and studies in plasma from healthy individuals showed that RvD6 is a biomarker that decreases with aging (50). RvD6 is also released from stem cells isolated from human periodontal ligaments, which is important in tissue regeneration (51). However, RvD6 is not detected in normal human tears (52). Compared to treatments with PEDF+DHA and RvD6, the new RvD6i accelerated corneal wound healing, and sensitivity, demonstrating a higher bioactivity (FIG. 16 Panels A and B).

Use of DHA for Dry Eye Disease.

DED affects between 5% and 40% of adults older than 40 years (53, 54) with an estimated 16.4 million people impacted in the United States (55). In a recent Dry Eye Workshop (DEWS II), dry eye was defined as “a multifactorial disease of ocular surface characterized by a loss of homeostasis of the tear film, and accompanied by ocular symptoms, in which tear film instability and hyperosmolarity, ocular surface inflammation and damage, and neurosensory abnormalities have etiologic roles” (54).

Within the last decade, there has been a number of clinical trials of DED patients with different etiologies using ω-3 fatty acids DHA and EPA supplementation with the argument that dietary fatty acids can be incorporated in the lacrimal gland as well as in plasma phospholipids (56). However, the effect of oral PUFA supplementation in DED is controversial. While some studies showed improvement, others showed insignificant effects. In Table 2, we summarized clinical trials conducted in the last ten years in which supplementation with DHA was used to treat DED of different etiologies.

TABLE 2
Summary of clinical trials in the last 10 years for DED using ω-3 FAs treatment.

| Study | Number of patients/treatment | Randomized | Masking | Effect | Comments |
|---|---|---|---|---|---|
| Brignole- | Dry eye, n = 127, time = 90 days | Yes/Yes | Double | No | Only decrease in the |
| Baudouin et al., | Group 1, n = 61 | | | Significant | percentage of HLA- |
| 2011 (57) | 142.5 mg EPA, 95 mg DHA, and | | | Effect | DR-positive cell |
| | supplements, 3× daily | | | | was detected in |
| | Group 2, n = 66 | | | | treated group. |
| | Placebo, medium-chain triglyceride, | | | | |
| | 3× daily | | | | |
| Wojtowicz et al., | Dry eye, n = 36, time = 90 days | Yes/Yes | Double | No | No changes in |
| 2011 (58) | Group 1 | | | Significant | aqueous tear |
| | 450 mg of EPA and 300 mg of DHA | | | Effect | evaporation. |
| | and 1000 mg of flaxseed oi, 1× daily | | | | |
| | Group 2 | | | | |
| | Placebo, wheat germ oil | | | | |
| Bhargava et al., | Dry eye, n = 528, time = 3 months | Yes/Yes | Double | Improved | |
| 2013 (59) | Group 1, n = 264 | | | | |
| | 325 mg EPA and 175 mg DHA, | | | | |
| | 2× daily Group 2, n = 254 | | | | |
| | Placebo, 2× daily | | | | |
| Kangari et al., | Dry eye, n = 64, time = 30 days | Yes | Double | Slightly | |
| 2013 (60) | Group 1, n = 33 | | | Improved | |
| | 180 mg EPA and 120 mg DHA, 2× | | | | |
| | daily Group 2, n = 31 | | | | |
| | Placebo, medium-chain triglyceride | | | | |
| Olenik et al., | Meibomian gland dysfunction, n = 64, | Yes/No | Double | Slightly | No significant |
| 2013 (61) | time = 3 months | | | Improved | differences in |
| | Group 1, n = 33 | | | | comeal staining |
| | Brudysec (350 mg DHA, 42.5 mg EPA, | | | | from placebo. |
| | 30 mg DPA), 3× daily | | | | |
| | Group 2, n = 31 | | | | |
| | Placebo, 500 mg sunflower oil, 3× daily | | | | |
| | All patient received cleaning the lid | | | | |
| | margins with neutral baby shampoo and | | | | |
| | artificial tears without preservatives | | | | |
| Ong et al., | Healthy photorefiractive keratectomy | Yes/Yes | Single | Improved | Treatment was 2 |
| 2013 (62) | (PRK) patients, n = 18, time = 6 weeks | | | | weeks before PRK |
| | Group 1, n = 9 | | | | surgery through 1 |
| | 250 mg EPA and DHA), 333 mg of | | | | month after surgery. |
| | flaxseed oil, and 61 IU vitamin E, | | | | |
| | 3× daily | | | | |
| | Group 2, n = 9 Control | | | | |
| Sheppard et al., | Post-menopausal women with dry eye, | Yes/Yes | Double | Improved | Placebo treatment |
| 2013 (63) | n = 38, time = 6 months | | | | also increased |
| | Group 1, n = 19 | | | | HLA-DR intensity |
| | 49 mg ALA, 31.5 mg EPA, 3.75 mg | | | | by 36% ± 9% and |
| | DPA, 25 | | | | CD11c by 34% ± |
| | mg DHA, 177.5 mg LA, 60 mg GLA, | | | | 7% when compared |
| | <0.75 | | | | with supplement |
| | AA, and supplements, 4× daily | | | | treatment. |
| | Group 2, n = 19 | | | | |
| | Placebo | | | | |
| Olenik et al., | Dry eye, n = 905, time = 12 weeks | No/No | No | Improved | A total of 68.1% of |
| 2014 (64) | Brudysec (350 mg DHA, 42,5 mg EPA, | | | | patients reported |
| | 30 mg DPA), 3× daily. | | | | better tolerance to |
| | No control of placebo | | | | contact lenses after |
| | | | | | treatment. |
| Georgakopoulos | Diabetic Patients with Dry Eye, | No/No | No | Improved | |
| et al., 2015 | time = 3 months | | | | |
| (65) | Group 1, n = 36 | | | | |
| | 170 mg EPA and 115 mg DHA, | | | | |
| | 3× daily | | | | |
| Bhargava et al., | Computer-related dry eye, n = 456, | Yes/Yes | Double | Improved | |
| 2015 (66) | time = 3 months | | | | |
| | Group 1, n = 220 | | | | |
| | 180 mg EPA and 120 mg DHA, | | | | |
| | 2× daily Group 2, n = 236 | | | | |
| | Placebo containing olive oil, 2× daily | | | | |
| | Baseline-T0 | | | | |
| | 1 month of treatment-T1 2 months of | | | | |
| | treatment-T2 3 months of treatment-T3 | | | | |
| Deinema, | Dry eye, n = 54, time = 3 months | Yes/Yes | Double | Slightly | Both krill and fish |
| 2017 (67) | Group 1, n = 18 | | | Improved | oil moderated |
| | krill oil (945 mg/day EPA + 510 | | | | reduce the dry eye |
| | mg/day DHA) | | | | symptoms. |
| | Group 2, n = 19 | | | | The |
| | fish oil (1000 mg/day EPA + 500 | | | | proinflammatory |
| | mg/day DHA) | | | | cytokine interleukin |
| | Group 3, n =17 | | | | 17A was |
| | placebo (olive oil, 1500 mg/day) | | | | significantly |
| | | | | | reduced in the krill |
| | | | | | oil group only at |
| | | | | | day 90. |
| Goyal et al., | LASIK patients, n = 60, time = 12 | Yes/Yes | Double | Slightly | Less eyes had |
| 2017 (68) | weeks Group 1, n = 30 | | | Improved | conjunctival |
| | 180 mg of EPA and 120 mg of DHA, | | | | staining with |
| | 4× daily Group 2, n = 3 | | | | Lissamine. |
| | Placebo | | | | |
| DREAM, | Dry eye, n = 499, time = 12 months | Yes/Yes | Double | No | Significantly |
| 2019 (69) | Group 1, n = 329 | | | Significant | increased EPA and |
| | 400 mg of EPA and 200 mg of DHA, | | | Effect | DHA in red blood |
| | 5× daily Group 2, n = 170 | | | | cells. |
| | Placebo, 1000 mg of refined olive oil, | | | | |
| | 5x daily | | | | |
| Fogt et al., | Dry eye, n = 19, time = 1 hours Drug 1, | Yes/Yes | Double | Improved | The lipid layer |
| 2019 (70) | n = 19 | | | (short | thickness (LLT) |
| | Refresh Optive plus Omega3 flaxseed | | | time) | was increased from |
| | oil Drug 2, n = 19 | | | | baseline at 15 mins |
| | Refresh Optive | | | | for both treatments. |
| | The drug is randomly picked for two | | | | Only Refresh |
| | different visits (>2 days between) | | | | Optive plus |
| | | | | | Omega3 patients |
| | | | | | had higher LLT up |
| | | | | | to 1 hour after |
| | | | | | instillation. |

The underline indicates the clinical trial using topical eye drops.

One of the most important trials, the DREAM study, which involved a total of 499 patients with 329 receiving 12 months of supplementation with EPA and DHA and 170 patients treated with refined olive oil as a placebo (69), indicated that there was no improvement. This study increases the doubtfulness about the benefit of DHA in the treatment of DED. For this reason, in this review, we point out problems that may explain the results of DHA supplementation.

One concern is the form of DHA supplementation. Most of the studies employed natural, enriched fish oil. However, analysis of fish oil composition showed that the PUFAs are mainly esterified in triglycerides. DHA from the diet needs to be taken up by the liver before being esterified in the sn-2 position of membrane phospholipid, mainly PC (71). DHA-phospholipids are then packaged in very-low-density lipoproteins (VLDLs) or other lipoproteins before being released into the blood stream (71,72). Therefore, supplementation of DHA or EPA from fish oil reaches the ocular surface, especially the cornea, is very low. This is supported by previous studies where krill oil, which mainly contains PC with long chain PUFAs, showed a higher absorption rate in rat blood and brain than fish oil (73). There is only one study that uses krill oil to treat DED, a small clinical trial (18 participants per group) in which Deinema and colleagues showed lower Ocular Surface Disease Index and IL-17A levels in krill oil supplementation than in fish oil after 90 days of treatment (67) and Table 2.

In addition, it is important to note that the cornea is avascular, therefore, dietary fatty acids incorporated into the corneal cellular membrane is unlikely. This is supported by a study using ¹⁴C-labeled DHA given orally to rats, which showed a very small rate (less than 0.03% of the oral dose) of DHA that reached the eye compartment (74). Of this quantity, the amount that might get into the cornea is very low since the retina takes most of the DHA from sub-retinal blood vessels. Therefore, PUFA enrichment in the lacrimal gland is insufficient to ensure a beneficial treatment in the cornea.

To our knowledge, there is only one clinical trial using topical DHA ((70) and Table 2).

This trial was based on previous studies showing that AA, DHA, and EPA were found in the tears of patients with DED and that the ratio of ω-6 (AA):ω-3 (DHA+EPA) correlates with the severity of the tear film dysfunction (75). The small trial (19 patients treated topically with DHA) demonstrated that treatment with eye drops containing omega-3 fatty acids increases lipid layer thickness of the tear film up to 1 hour after instillation (70).

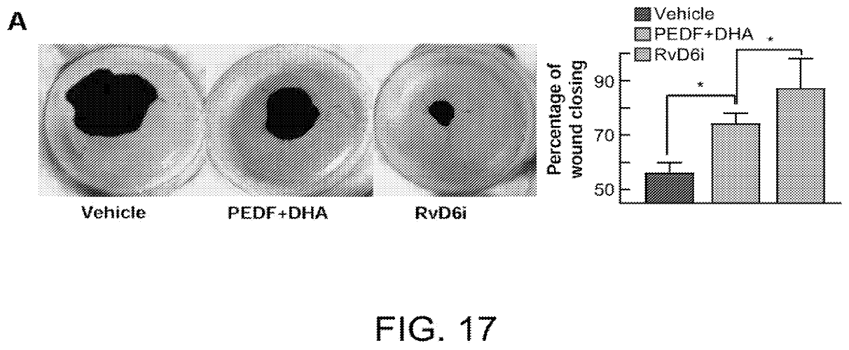

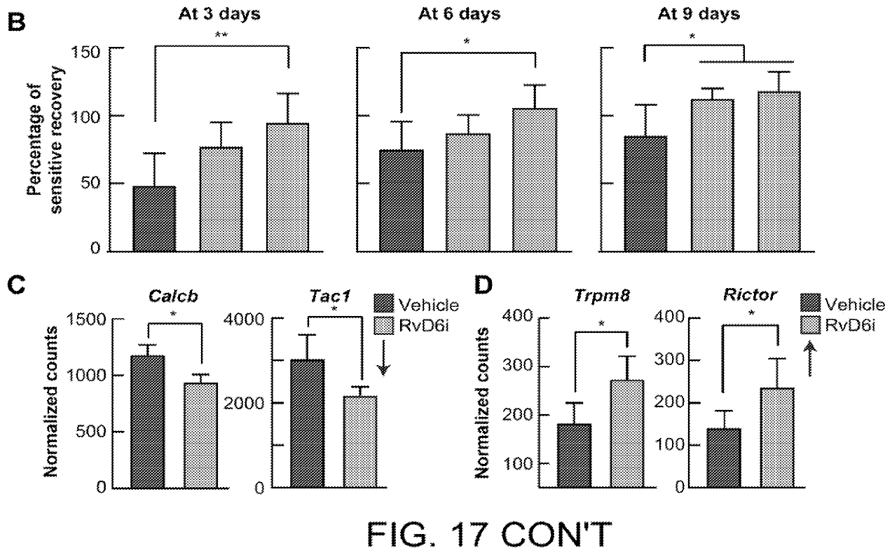

Lastly, our animal studies show that DHA is rapidly incorporated in the corneal phospholipids, mainly in PE and PC, to increase nerve density. Decrease in nerve density is a well-documented alteration in DED that requires both PEDF and DHA to regenerate the nerves. The treatment releases DHA and stimulates the synthesis of RvD6i, and this docosanoid increases wound healing and sensitivity (FIG. 17 Panels A and B) and, without wishing to be bound by theory, is of better therapeutic use than DHA for DED (40).

The effectiveness of docosanoids in decreasing inflammation and increasing corneal wound healing, nerve regeneration, and tear secretion has been demonstrated clearly on several different models of injury, infection, diabetes, corneal angiogenesis, and transplantation (Table 3). These results emphasized the action of docosanoids as potent drugs.

TABLE 3
In vivo studies using PEDF + DHA or docosanoids for corneal damages.

| Animal/model | Docosanoids | Administration | Key Result |
|---|---|---|---|
| Mouse | NPD1 | Topical eye drops, | Increased the rate of re-epithelialization |
| Comeal epithelium | 17S-HDHA | 3× daily for 96 | Increased PMNs in the cornea Decreased |
| removal up to the | | hours | formation of the proinflammatory chemokine |
| corneal/limbal | | | KC |
| border 2005 (76) | | | |
| Mouse | RvD1 | Subconjunctival | Reduced numbers of infiltrating neutrophils and |
| Suture-induced | | injection, every 48 | macrophages and reduced mRNA expression |
| inflammatory | | hours | levels of TNF-α, IL-1α, IL-1β, VEGF-A, |
| comeal angiogenesis | | Time = 14 days | VEGF-C, and VEGFR2. |
| 2009 (77) | | | Suppressed suture-induced or IL-1β-induced |
| | | | hemangiogenesis (HA) but not |
| | | | lymphangiogenesis. |
| Rabbit Experimental | PEDF + DHA | Topical using | Increased nerve density and tear secretion in |
| PRK 2010 (19), | PEDF | collagen shield, | treated group for 8 weeks with PEDF + DHA, |
| 2012 (20), | domains + DHA | twice a week | NPD1 synthesis peaked at 1 week and was four |
| 2015 (21) | | Time = 8 weeks | times higher in the PEDF + DHA-treated group |
| | | | than in the controls. |
| | | | The 44-mer domain of PEDF is more potent |
| | | | than 34-mer domain |
| Rabbit Experimental | NPD1 | Topical eye drops | Increased subepithelial corneal nerves and tear |
| PRK 2013 (41) | | of NPD1 (33 ng/eye) | secretion. |
| | | 3× daily for 6 weeks | Decreased neutrophil infiltration after 2 and 4 |
| | | | days of treatment |
| Mouse Corneal | RvD1 | Intravenous | Reduced allosensitization. |
| allotransplantation | analogue | injection | Reduced angiogenesis at the graft site Enhanced |
| 2014 (78) | | | graft survival. |
| Mouse | RvD1 RvD1- | Daily i.p. injections | Reduced the diabetes-induced comeal nerve |
| Type 2 diabetes | methyl ester | of 1 ng/g body | lost. |
| 2017 (79) | RvD2- | weight for 8 weeks | Methyl ester version is less bioactive than free |
| | methyl ester | | fatty acid. |
| Rabbit HSV1 | PEDF + DHA | Topical eye drops, | Stronger infiltration of CD4+ T cells, neutrophils |
| corneal | | 3× daily for 2 weeks. | and macrophages at 7-days, then decreased by |
| infection 2017 (22) | | Topical using | 14 days |
| | | collagen shield, | Corneal nerve density increased at 12-weeks |
| | | twice a week for 10 | with functionl recovery of corneal sensation |
| | | weeks more | |
| Mouse | PEDF + DHA | Topical eye drops, | Increase in corneal epithelial nerve |
| Type 1 diabetes | | 3× daily for 14 days | regeneration, substance P-positive nerve density |
| Corneal epithelium | | | and tear volume. |
| removal inside 2 mm | | | Accelerated corneal wound healing, selectively |
| diameter central | | | recruited type 2 macrophages, and prevented |
| area 2017 (23) | | | neutrophil infiltration |
| Mouse | PEDF + DHA | Topical eye drops, | Increased nerve regeneration and tear secretion. |
| Corneal nerve | | 3× daily for 7 days | Phospholipase A2 activity of the PEDF- |
| cutting 2017 (27) | | | receptor (PEDF-R) is required for the working |
| | | | mechanism |
| Mouse | RvD1 | Topical eye drops, | Promotes corneal epithelial wound healing and |
| Type 1 diabetes | | 4× daily for 14 days | nerve regeneration |
| Corneal epithelium | | | |
| removal inside 2 mm | | | |
| diameter central | | | |
| area 2018 (80) | | | |
| Mouse | RvD6i | Topical eye drops, | Discovered the RvD6i underlying the |
| Corneal epithelium | | 3× daily for 12 days | mechanism of PEDF + DHA |
| removal inside 2 mm | | | Increased corneal wound healing, sensitivity |
| diameter central | | | and nerve regeneration. Reduced inflammatory- |
| area 2020 (40) | | | and pain-related neuropeptides, increase ion |
| | | | channel gene expression in TG |

Underlined indicates studies from our laboratory.

RvD6i Regulates Genes Involved in Neurogenesis and Pain in the TG

Previous studies have showed that cornea treatment with PEDF and DHA also stimulated the synthesis of the docosanoid NPD1. However, the synthetized amount is much lower than RvD6i (19, 40). When adding NPD1 to injured corneas, there is an increase in gene expression and protein levels of the neurotrophins NGF, brain-derived neurotrophic factor (BDNF), and semaphorin A2 (Sema7A) that stimulate axon growth (27). These proteins are secreted into tears and activate receptors in the corneal nerve terminals to facilitate downstream signaling as well as retrograde to the neurons of the TG.

Using RNA-sequencing to analyze the gene expression in TG from the injured corneas of mice, we reveal that the product of PEDF+DHA, RvD6i, applied topically to the cornea induces the expression of two interesting genes in the TG, chromosome 9 open reading frame 72 (C9orf72), and glycoprotein MGA (Gpm6A) (40). These genes stimulate neurogenesis and growth cone formation (81,82).

Ocular pathologies that damage corneal nerves in many cases produce neuropathic pain (83). In addition, there are a significant number of patients who have symptoms of DED and experience neuropathic pain, indicating that there is an active cornea-TG relationship (84). Two genes involved in pain were decreased in corneas treated with RvD6i: Tac1 that encodes substance P (SP), which is one of the most abundant neuropeptides expressed in corneal nerves (4, 85, 86), and Calcb, which encodes Calcitonin gene-related peptide (CGRP) (also abundant in corneal nerves) (4,20) (FIG. 17 Panel C). Both neuropeptides have important roles in neurogenic inflammation and pain (87, 88). In addition, corneal treatment with RvD6i increased the gene expression of transient receptor potential melastatin 8 (Trmp8) (FIG. 17 Panel D). TRPM8 ion channels are cool sensors that regulate the wetting of the ocular surface and produce an analgesic effect on chronic pain (89-93). Our studies in a mouse model where the nerves had been damaged at the level of the anterior stroma, showed that cornea TRPM8-positive nerve fibers only reach 50% of their normal density after 3 months of injury, indicating that the decrease in TRPM8 may contribute to DE-like pain (94). Therefore, decreased expression of SP and CGRP and increased expression of TRPM8 after injury and treatment with RvD6i indicates that the new docosanoid could protect corneas from pain. It also provides compositions and methods for treating ocular surface damage, such as corneal neurotrophic ulcers, since studies have shown ocular pain as a side effect of increased corneal nerve regeneration caused by topical treatment with NGF (95).Studies using RvD1 and RvD5 had shown pain attenuation in a mouse model of tibia bone fracture, while RvD3 and RvD4 had no effect (96). These differences could be due to different expression of its receptors. In an osteoporosis mouse model the precursor of RvDs 17R-hydroxy DHA decrease pain behavior probably trough activation of AXL receptors (97). Another important finding is that RvD6i is a strong inducer of the gene expression of Rictor in the TG (40) (FIG. 17 Panel D). RICTOR is a key component of the mammalian target of rapamycin-insensitive complex 2 (mTORC2) and plays a role in anti-inflammation and axon growth of sensory neurons after injury (98).

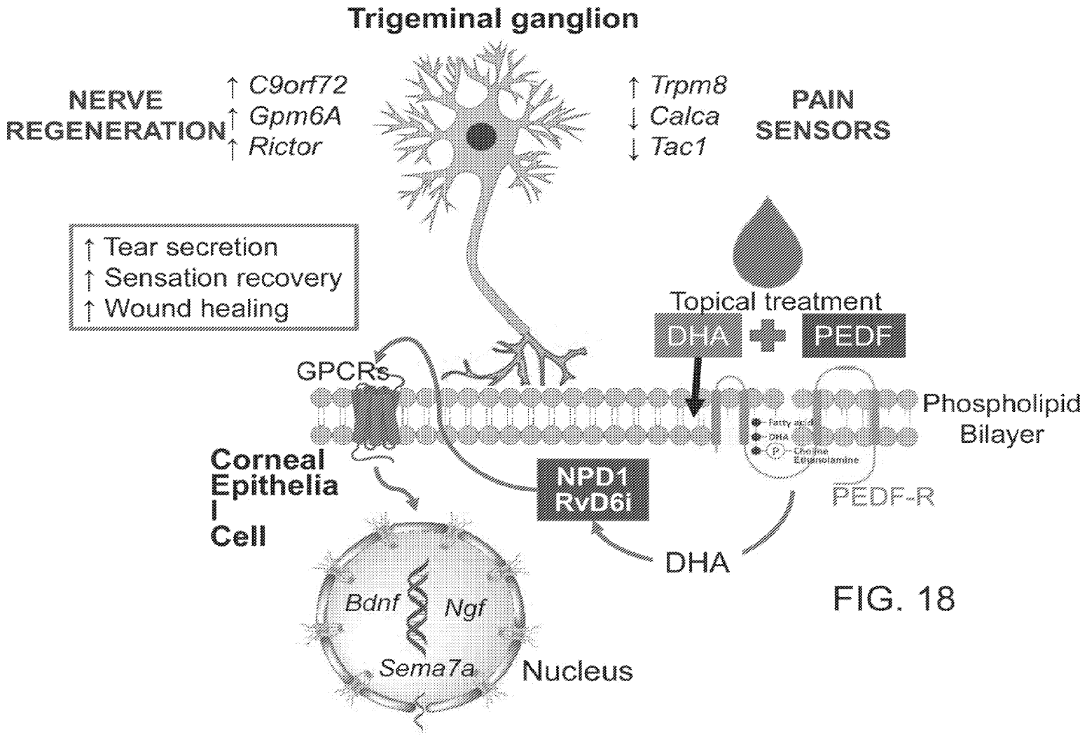

A summarized scheme of the signaling pathways of docosanoids stimulated by PEDF and DHA is shown in FIG. 18.

### CONCLUSIONS

Cornea innervation plays a pivotal role in maintaining the homeostasis of the ocular surface and tissue clarity (7). Damage to corneal nerves produces a decrease in tear production and blinking reflex and can impair epithelial wound healing resulting in loss of transparency and vision (8-13). Therefore, better knowledge on corneal nerve function and repair will increase therapeutic strategies for pathologies that affect corneal innervation. Without wishing to be bound by theory, DHA-derived docosanoids, such as the new mediator RvD6i, are treatments to reduce cornea-related inflammation. The effect of this lipid in accelerating nerve regeneration and modulating the gene expression of components of neuropathic pain in the TG could provide a new alternative in the treatment of patients with DE following refractive surgery as well as co-treatment to several pathologies that decrease corneal nerve density. Prospective human clinical trials can be to validate optimal dosing, modes of administration, efficacy, and safety of these new treatments for DE and ocular surface diseases.

### REFERENCES CITED IN THIS EXAMPLE

DelMonte, D. W., and T. Kim. 2011. Anatomy and physiology of the cornea. J. Cataract Refract. Surg. 37: 588-598.
Meek, K. M., and C. Knupp. 2015. Corneal structure and transparency. Prog. Retin. Eye Res. 49: 1-16.
Hanna, C., D. S. Bicknell, and J. E. O′brien. 1961. Cell turnover in the adult human eye. Arch. Ophthalmol. Chic. Ill. 1960. 65: 695-698.
Müller, L. J., C. F. Marfurt, F. Kruse, and T. M. T. Tervo. 2003. Corneal nerves: structure, contents and function. Exp. Eye Res. 76: 521-542.
He, J., N. G. Bazan, and H. E. P. Bazan. 2010. Mapping the entire human corneal nerve architecture. Exp. Eye Res. 91: 513-523.
Al-Aqaba, M. A., V. K. Dhillon, I. Mohammed, D. G. Said, and H. S. Dua. 2019. Corneal nerves in health and disease. Prog. Retin. Eye Res. 73: 100762.
Shaheen, B. S., M. Bakir, and S. Jain. 2014. Corneal nerves in health and disease. Surv. Ophthalmol. 59: 263-285.
He, J., and H. E. P. Bazan. 2012. Mapping the nerve architecture of diabetic human corneas. Ophthalmology. 119: 956-964.
Hamrah, P., A. Cruzat, M. H. Dastjerdi, L. Zheng, B. M. Shahatit, H. A. Bayhan, R. Dana, and D. Pavan-Langston. 2010. Corneal sensation and subbasal nerve alterations in patients with herpes simplex keratitis: an in vivo confocal microscopy study. Ophthalmology. 117: 1930-1936.
Cruzat, A., D. Witkin, N. Baniasadi, L. Zheng, J. B. Ciolino, U. V. Jurkunas, J. Chodosh, D. Pavan-Langston, R. Dana, and P. Hamrah. 2011. Inflammation and the nervous system: the connection in the cornea in patients with infectious keratitis. Invest. Ophthalmol. Vis. Sci. 52: 5136-5143.
He, J., and H. E. P. Bazan. 2013. Corneal nerve architecture in a donor with unilateral epithelial basement membrane dystrophy. Ophthalmic Res. 49: 185-191.
Pham, T. L., A. Kakazu, J. He, and H. E. P. Bazan. 2018. Mouse strains and sexual divergence in corneal innervation and nerve regeneration. FASEB J. Off. Publ. Fed. Am. Soc. Exp. Biol. fj201801957R.
Garcia-Gonzalez, M., P. Cañadas, J. Gros-Otero, I. Rodriguez-Perez, R. Cañones-Zafra, V. Kozobolis, and M. A. Teus. 2019. Long-term corneal subbasal nerve plexus regeneration after laser in situ keratomileusis. J. Cataract Refract. Surg. 45: 966-971.
Esquenazi, S., H. E. P. Bazan, V. Bui, J. He, D. B. Kim, and N. G. Bazan. 2005. Topical Combination of NGF and DHA Increases Rabbit Corneal Nerve Regeneration after Photorefractive Keratectomy. Invest. Ophthalmol. Vis. Sci. 46: 3121-3127.
Mukherjee, P. K., V. L. Marcheselli, C. N. Serhan, and N. G. Bazan. 2004. Neuroprotectin D1: a docosahexaenoic acid-derived docosatriene protects human retinal pigment epithelial cells from oxidative stress. Proc. Natl. Acad. Sci. U S. A. 101: 8491-8496.
Mukherjee, P. K., V. L. Marcheselli, S. Barreiro, J. Hu, D. Bok, and N. G. Bazan. 2007. Neurotrophins enhance retinal pigment epithelial cell survival through neuroprotectin D1 signaling. Proc. Natl. Acad. Sci. 104: 13152-13157.
Tombran-Tink, J., and C. J. Barnstable. 2003. PEDF: a multifaceted neurotrophic factor. Nat. Rev. Neurosci. 4: 628-636.
Kenchegowda, S., J. He, and H. E. P. Bazan. 2013. Involvement of pigment epithelium-derived factor, docosahexaenoic acid and neuroprotectin D1 in corneal inflammation and nerve integrity after refractive surgery. Prostaglandins Leukot. Essent. Fat. Acids PLEFA. 88: 27-31.
Cortina, M. S., J. He, N. Li, N. G. Bazan, and H. E. P. Bazan. 2010. Neuroprotectin D1 Synthesis and Corneal Nerve Regeneration after Experimental Surgery and Treatment with PEDF plus DHA. Invest. Ophthalmol. Vis. Sci. 51: 804-810.
Cortina, M. S., J. He, N. Li, N. G. Bazan, and H. E. P. Bazan. 2012. Recovery of corneal sensitivity, calcitonin gene-related peptide-positive nerves, and increased wound healing induced by pigment epithelial-derived factor plus docosahexaenoic acid after experimental surgery. Arch. Ophthalmol. Chic. Ill. 1960. 130: 76-83.
He, J., M. S. Cortina, A. Kakazu, and H. E. P. Bazan. 2015. The PEDF Neuroprotective Domain Plus DHA Induces Corneal Nerve Regeneration After Experimental Surgery. Invest. Ophthalmol. Vis. Sci. 56: 3505-3513.
He, J., D. Neumann, A. Kakazu, T. L. Pham, F. Musarrat, M. S. Cortina, and H. E. P. Bazan. 2017. PEDF plus DHA modulate inflammation and stimulate nerve regeneration after HSV-1 infection. Exp. Eye Res. 161: 153-162.
He, J., T. L. Pham, A. Kakazu, and H. E. P. Bazan. 2017. Recovery of Corneal Sensitivity and Increase in Nerve Density and Wound Healing in Diabetic Mice After PEDF Plus DHA Treatment. Diabetes. 66: 2511-2520.
Houenou, L. J., A. P. D'Costa, L. Li, V. L. Turgeon, C. Enyadike, E. Alberdi, and S. P. Becerra. 1999. Pigment epithelium-derived factor promotes the survival and differentiation of developing spinal motor neurons. J. Comp. Neurol. 412: 506-514.
Amaral, J., and S. P. Becerra. 2010. Effects of Human Recombinant PEDF Protein and PEDF-Derived Peptide 34-mer on Choroidal Neovascularization. Invest. Ophthalmol. Vis. Sci. 51: 1318-1326.
Notari, L., V. Baladron, J. D. Aroca-Aguilar, N. Balko, R. Heredia, C. Meyer, P. M. Notario, S. Saravanamuthu, M.-L. Nueda, F. Sanchez-Sanchez, J. Escribano, J. Laborda, and S. P. Becerra. 2006. Identification of a lipase-linked cell membrane receptor for pigment epithelium-derived factor. J. Biol. Chem. 281: 38022-38037.
Pham, T. L., J. He, A. H. Kakazu, B. Jun, N. G. Bazan, and H. E. P. Bazan. 2017. Defining a mechanistic link between pigment epithelium-derived factor, docosahexaenoic acid, and corneal nerve regeneration. J. Biol. Chem. 292: 18486-18499.
Broekhuyse, R. M. 1968. Phospholipids in tissues of the eye. I. Isolation, characterization and quantitative analysis by two-dimensional thin-layer chromatography of diacyl and vinyl-ether phospholipids. Biochim. Biophys. Acta. 152: 307-315.
Tschetter, R. T. 1966. Lipid analysis of the human cornea with and without arcus senilis. Arch. Ophthalmol. Chic. Ill. 1960. 76: 403-405.
Bazan, H. E., and N. G. Bazan. 1984. Composition of phospholipids and free fatty acids and incorporation of labeled arachidonic acid in rabbit cornea. Comparison of epithelium, stroma and endothelium. Curr. Eye Res. 3: 1313-1319.
Katsura, K., E. B. Rodriguez de Turco, B. K. Siesjó, and N. G. Bazan. 2004. Effects of hyperglycemia and hypercapnia on lipid metabolism during complete brain ischemia. Brain Res. 1030: 133-140.
Rodriguez de Turco, E. B., L. Belayev, Y. Liu, R. Busto, N. Parkins, N. G. Bazan, and M. D. Ginsberg. 2002. Systemic fatty acid responses to transient focal cerebral ischemia: influence of neuroprotectant therapy with human albumin. J. Neurochem. 83: 515-524.
Bazan, H. E., D. L. Birkle, R. Beuerman, and N. G. Bazan. 1985. Cryogenic lesion alters the metabolism of arachidonic acid in rabbit cornea layers. Invest. Ophthalmol. Vis. Sci. 26: 474-480.
Bazan, H. E., Y. Tao, M. A. DeCoster, and N. G. Bazan. 1997. Platelet-activating factor induces cyclooxygenase-2 gene expression in corneal epithelium. Requirement of calcium in the signal transduction pathway. Invest. Ophthalmol. Vis. Sci. 38: 2492-2501.
Liclican, E. L., V. Nguyen, A. B. Sullivan, and K. Gronert. 2010. Selective Activation of the Prostaglandin E2 Circuit in Chronic Injury-Induced Pathologic Angiogenesis. Invest. Ophthalmol. Vis. Sci. 51: 6311-6320.
Amico, C., M. Yakimov, M. V. Catania, R. Giuffrida, M. Pistone, and V. Enea. 2004. Differential expression of cyclooxygenase-1 and cyclooxygenase-2 in the cornea during wound healing. Tissue Cell. 36: 1-12.
Hurst, J. S., M. Balazy, H. E. Bazan, and N. G. Bazan. 1991. The epithelium, endothelium, and stroma of the rabbit cornea generate (12S)-hydroxyeicosatetraenoic acid as the main lipoxygenase metabolite in response to injury. J. Biol. Chem. 266: 6726-6730.
Sharma, G. D., P. Ottino, N. G. Bazan, and H. E. P. Bazan. 2005. Epidermal and hepatocyte growth factors, but not keratinocyte growth factor, modulate protein kinase Calpha translocation to the plasma membrane through 15(S)-hydroxyeicosatetraenoic acid synthesis. J. Biol. Chem. 280: 7917-7924.
Leedom, A. J., A. B. Sullivan, B. Dong, D. Lau, and K. Gronert. 2010. Endogenous LXA4 Circuits Are Determinants of Pathological Angiogenesis in Response to Chronic Injury. Am. J. Pathol. 176: 74-84.
Pham, T. L., A. H. Kakazu, J. He, B. Jun, N. G. Bazan, and H. E. P. Bazan. 2020. Novel RvD6 stereoisomer induces corneal nerve regeneration and wound healing post-injury by modulating trigeminal transcriptomic signature. Sci. Rep. 10: 1-12.
Cortina, M. S., J. He, T. Russ, N. G. Bazan, and H. E. P. Bazan. 2013. Neuroprotectin D1 Restores Corneal Nerve Integrity and Function After Damage From Experimental Surgery. Invest. Ophthalmol. Vis. Sci. 54: 4109-4116.
Bazan, N. G. 2018. Docosanoids and elovanoids from omega-3 fatty acids are pro-homeostatic modulators of inflammatory responses, cell damage and neuroprotection. Mol. Aspects Med. 64: 18-33.
Serhan, C. N., and B. D. Levy. 2018. Resolvins in inflammation: emergence of the pro-resolving superfamily of mediators. J. Clin. Invest. 128: 2657-2669.
Krishnamoorthy, S., A. Recchiuti, N. Chiang, S. Yacoubian, C.-H. Lee, R. Yang, N. A. Petasis, and C. N. Serhan. 2010. Resolvin D1 binds human phagocytes with evidence for proresolving receptors. Proc. Natl. Acad. Sci. U S. A. 107: 1660-1665.
Chiang, N., J. Dalli, R. A. Colas, and C. N. Serhan. 2015. Identification of resolvin D2 receptor mediating resolution of infections and organ protection. J. Exp. Med. 212: 1203-1217.
Dalli, J., J. W. Winkler, R. A. Colas, H. Arnardottir, C.-Y. C. Cheng, N. Chiang, N. A. Petasis, and C. N. Serhan. 2013. Resolvin D3 and Aspirin-Triggered Resolvin D3 Are Potent Immunoresolvents. Chem. Biol. 20: 188-201.
Chiang, N., G. Fredman, F. Backhed, S. F. Oh, T. Vickery, B. A. Schmidt, and C. N. Serhan. 2012. Infection regulates pro-resolving mediators that lower antibiotic requirements. Nature. 484: 524-528.
Bang, S., Y.-K. Xie, Z.-J. Zhang, Z. Wang, Z.-Z. Xu, and R.-R. Ji. 2018. GPR37 regulates macrophage phagocytosis and resolution of inflammatory pain. J. Clin. Invest. 128: 3568-3582.
Chiang, N., S. Libreros, P. C. Norris, X. de la Rosa, and C. N. Serhan. 2019. Maresin 1 activates LGR6 receptor promoting phagocyte immunoresolvent functions. J. Clin. Invest. 129: 5294-5311.
Jové, M., I. Maté, A. Naudi, N. Mota-Martorell, M. Portero-Otin, M. De la Fuente, and R. Pamplona. 2016. Human Aging Is a Metabolome-related Matter of Gender. J. Gerontol. Biol. Sci. Med. Sci. 71: 578-585.
Cianci, E., A. Recchiuti, O. Trubiani, F. Diomede, M. Marchisio, S. Miscia, R. A. Colas, J. Dalli, C. N. Serhan, and M. Romano. 2016. Human Periodontal Stem Cells Release Specialized Proresolving Mediators and Carry Immunomodulatory and Prohealing Properties Regulated by Lipoxins. Stem Cells Transl. Med. 5: 20-32.
English, J. T., P. C. Norris, R. R. Hodges, D. A. Dartt, and C. N. Serhan. 2017. Identification and Profiling of Specialized Pro-Resolving Mediators in Human Tears by Lipid Mediator Metabolomics. Prostaglandins Leukot. Essent. Fatty Acids. 117: 17-27.
The epidemiology of dry eye disease: report of the Epidemiology Subcommittee of the International Dry Eye WorkShop (2007). 2007. Ocul. Surf 5: 93-107.
Stapleton, F., M. Alves, V. Y. Bunya, I. Jalbert, K. Lekhanont, F. Malet, K.-S. Na, D. Schaumberg, M. Uchino, J. Vehof, E. Viso, S. Vitale, and L. Jones. 2017. TFOS DEWS II Epidemiology Report. Ocul. Surf 15: 334-365.
Farrand, K. F., M. Fridman, I. Ö. Stillman, and D. A. Schaumberg. 2017. Prevalence of Diagnosed Dry Eye Disease in the United States Among Adults Aged 18 Years and Older. Am. J. Ophthalmol. 182: 90-98.
Schnebelen, C., S. Viau, S. Grégoire, C. Joffre, C. P. Creuzot-Garcher, A. M. Bron, L. Bretillon, and N. Acar. 2009. Nutrition for the eye: different susceptibility of the retina and the lacrimal gland to dietary omega-6 and omega-3 polyunsaturated fatty acid incorporation. Ophthalmic Res. 41: 216-224.
Brignole-Baudouin, F., C. Baudouin, P. Aragona, M. Rolando, M. Labetoulle, P. J. Pisella, S. Barabino, R. Siou-Mermet, and C. Creuzot-Garcher. 2011. A multicentre, double-masked, randomized, controlled trial assessing the effect of oral supplementation of omega-3 and omega-6 fatty acids on a conjunctival inflammatory marker in dry eye patients. Acta Ophthalmol. (Copenh.). 89: e591-e597.
Wojtowicz, J. C., I. Butovich, E. Uchiyama, J. Aronowicz, S. Agee, and J. P. McCulley. 2011. Pilot, Prospective, Randomized, Double-masked, Placebo-controlled Clinical Trial of an Omega-3 Supplement for Dry Eye. Cornea. 30: 308-314.
Bhargava, R., P. Kumar, M. Kumar, N. Mehra, and A. Mishra. 2013. A randomized controlled trial of omega-3 fatty acids in dry eye syndrome. Int. J. Ophthalmol. 6: 811-816.
Kangari, H., M. H. Eftekhari, S. Sardari, H. Hashemi, J. Salamzadeh, M. Ghassemi-Broumand, and M. Khabazkhoob. 2013. Short-term Consumption of Oral Omega-3 and Dry Eye Syndrome. Ophthalmology. 120: 2191-2196.
Olenik, A., I. Jimenez-Alfaro, N. Alejandre-Alba, and I. Mahillo-Fernandez. 2013. A randomized, double-masked study to evaluate the effect of omega-3 fatty acids supplementation in meibomian gland dysfunction. Clin. Interv. Aging. 8: 1133-1138.
Ong, N., T. Purcell, A.-C. Roch-Levecq, D. Wang, M. Isidro, K. Bottos, C. Heichel, and D. Schanzlin. 2013. Epithelial Healing and Visual Outcomes of Patients Using Omega-3 Oral Nutritional Supplements Before and After Photorefractive Keratectomy: A Pilot Study. Cornea. 32: 761-765.
Sheppard, J., R. Singh, A. McClellan, M. Weikert, S. Scoper, T. Joly, W. Whitley, E. Kakkar, and S. Pflugfelder. 2013. Long-term Supplementation With n-6 and n-3 PUFAs Improves Moderate-to-Severe Keratoconjunctivitis Sicca: A Randomized Double-Blind Clinical Trial. Cornea. 32: 1297-1304.
Oleñik, A. 2014. Effectiveness and tolerability of dietary supplementation with a combination of omega-3 polyunsaturated fatty acids and antioxidants in the treatment of dry eye symptoms: Results of a prospective study. Clin. Ophthalmol. 8: 169-176.
Georgakopoulos, C. D., O. E. Makri, D. Pagoulatos, P. Vasilakis, P. Peristeropoulou, V. Kouli, M. I. Eliopoulou, and C. Psachoulia. 2017. Effect of Omega-3 Fatty Acids Dietary Supplementation on Ocular Surface and Tear Film in Diabetic Patients with Dry Eye. J. Am. Coll. Nutr. 36: 38-43.
Bhargava, R., P. Kumar, H. Phogat, A. Kaur, and M. Kumar. 2015. Oral omega-3 fatty acids treatment in computer vision syndrome related dry eye. Contact Lens Anterior Eye. 38: 206-210.
Deinema, L. A., A. J. Vingrys, C. Y. Wong, D. C. Jackson, H. R. Chinnery, and L. E. Downie. 2017. A Randomized, Double-Masked, Placebo-Controlled Clinical Trial of Two Forms of Omega-3 Supplements for Treating Dry Eye Disease. Ophthalmology. 124: 43-52.
Goyal, P., A. K. Jain, and C. Malhotra. 2017. Oral Omega-3 Fatty Acid Supplementation for Laser In Situ Keratomileusis-Associated Dry Eye. Cornea. 36: 169-175.
n-3 Fatty Acid Supplementation for the Treatment of Dry Eye Disease. 2018. N. Engl. J. Med. 378: 1681-1690.
Fogt, J. S., N. Fogt, P. E. King-Smith, H. Liu, and J. T. Barr. 2019. Changes in Tear Lipid Layer Thickness and Symptoms Following the Use of Artificial Tears with and Without Omega-3 Fatty Acids: A Randomized, Double-Masked, Crossover Study. Clin. Ophthalmol. Auckl. NZ. 13: 2553-2561.
Polozova, A., and N. Salem. 2007. Role of liver and plasma lipoproteins in selective transport of n-3 fatty acids to tissues: a comparative study of 14C-DHA and 3H-oleic acid tracers. J. Mol. Neurosci. MN. 33: 56-66.
Bazan, N. G., M. F. Molina, and W. C. Gordon. 2011. Docosahexaenoic Acid Signalolipidomics in Nutrition: Significance in Aging, Neuroinflammation, Macular Degeneration, Alzheimer's, and Other Neurodegenerative Diseases. Annu. Rev. Nutr. 31: 321-351.
Ahn, S. H., S. J. Lim, Y. M. Ryu, H.-R. Park, H. J. Suh, and S. H. Han. 2018. Absorption rate of krill oil and fish oil in blood and brain of rats. Lipids Health Dis. 17: 162.
Graf, B. A., G. S. M. J. E. Duchateau, A. B. Patterson, E. S. Mitchell, P. van Bruggen, J. H. Koek, S. Melville, and H. J. Verkade. 2010. Age dependent incorporation of 14C-DHA into rat brain and body tissues after dosing various 14C-DHA-esters. Prostaglandins Leukot. Essent. Fatty Acids. 83: 89-96.
Walter, S. D., K. Gronert, A. L. McClellan, R. C. Levitt, K. D. Sarantopoulos, and A. Galor. 2016. ω-3 Tear Film Lipids Correlate With Clinical Measures of Dry Eye. Invest. Ophthalmol. Vis. Sci. 57: 2472-2478.
Gronert, K., N. Maheshwari, N. Khan, I. R. Hassan, M. Dunn, and M. L. Schwartzman. 2005. A Role for the Mouse 12/15-Lipoxygenase Pathway in Promoting Epithelial Wound Healing and Host Defense. J. Biol. Chem. 280: 15267-15278.
Jin, Y., M. Arita, Q. Zhang, D. R. Saban, S. K. Chauhan, N. Chiang, C. N. Serhan, and R. Dana. 2009. Anti-angiogenesis Effect of the Novel Anti-inflammatory and Pro-resolving Lipid Mediators. Invest. Ophthalmol. Vis. Sci. 50: 4743-4752.
Hua, J., Y. Jin, Y. Chen, T. Inomata, H. Lee, S. K. Chauhan, N. A. Petasis, C. N. Serhan, and R. Dana. 2014. The Resolvin D1 Analogue Controls Maturation of Dendritic Cells and Suppresses Alloimmunity in Corneal Transplantation. Invest. Ophthalmol. Vis. Sci. 55: 5944-5951.
Obrosov, A., L. J. Coppey, H. Shevalye, and M. A. Yorek. 2017. Effect of Fish Oil vs. Resolvin D1, E1, Methyl Esters of Resolvins D1 or D2 on Diabetic Peripheral Neuropathy. J. Neurol. Neurophysiol. 8. [online] https://www.ncbi.nlm.nih.gov/pmc/articles/PMC5800519/ (Accessed Mar. 10, 2020).
Zhang, Z., X. Hu, X. Qi, G. Di, Y. Zhang, Q. Wang, and Q. Zhou. 2018. Resolvin D1 promotes corneal epithelial wound healing and restoration of mechanical sensation in diabetic mice. Mol. Vis. 24: 274-285.
Sivadasan, R., D. Homburg, C. Drepper, N. Frank, S. Jablonka, A. Hansel, X. Lojewski, J. Sterneckert, A. Hermann, P. J. Shaw, P. G. Ince, M. Mann, F. Meissner, and M. Sendtner. 2016. C9ORF72 interaction with cofilin modulates actin dynamics in motor neurons. Nat. Neurosci. 19: 1610-1618.
Formoso, K., M. D. Garcia, A. C. Frasch, and C. Scorticati. 2016. Evidence for a role of glycoprotein M6a in dendritic spine formation and synaptogenesis. Mol. Cell. Neurosci. 77: 95-104.
Goyal, S., and P. Hamrah. 2016. Understanding Neuropathic Corneal Pain—Gaps and Current Therapeutic Approaches. Semin. Ophthalmol. 31: 59-70.
Ferrari, G., F. Bignami, C. Giacomini, E. Capitolo, G. Comi, L. Chaabane, and P. Rama. 2014. Ocular Surface Injury Induces Inflammation in the Brain: In Vivo and Ex Vivo Evidence of a Corneal-Trigeminal Axis. Invest. Ophthalmol. Vis. Sci. 55: 6289-6300.
He, J., and H. E. P. Bazan. 2016. Neuroanatomy and Neurochemistry of Mouse Cornea. Invest. Ophthalmol. Vis. Sci. 57: 664-674.
He, J., T. L. Pham, and H. E. P. Bazan. 2019. Mapping the entire nerve architecture of the cat cornea. Vet. Ophthalmol. 22: 345-352.
Zieglgansberger, W. 2019. Substance P and pain chronicity. Cell Tissue Res. 375: 227-241.
Iyengar, S., M. H. Ossipov, and K. W. Johnson. 2017. The role of calcitonin gene-related peptide in peripheral and central pain mechanisms including migraine. Pain. 158: 543-559.
Belmonte, C., and J. Gallar. 2011. Cold Thermoreceptors, Unexpected Players in Tear Production and Ocular Dryness Sensations. Invest. Ophthalmol. Vis. Sci. 52: 3888-3892.
Parra, A., R. Madrid, D. Echevarria, S. del Olmo, C. Morenilla-Palao, M. C. Acosta, J. Gallar, A. Dhaka, F. Viana, and C. Belmonte. 2010. Ocular surface wetness is regulated by TRPM8-dependent cold thermoreceptors of the cornea. Nat. Med. 16: 1396-1399.
Proudfoot, C. J., E. M. Garry, D. F. Cottrell, R. Rosie, H. Anderson, D. C. Robertson, S. M. Fleetwood-Walker, and R. Mitchell. 2006. Analgesia mediated by the TRPM8 cold receptor in chronic neuropathic pain. Curr. Biol. CB. 16: 1591-1605.
Liu, B., L. Fan, S. Balakrishna, A. Sui, J. B. Morris, and S.-E. Jordt. 2013. TRPM8 is the Principal Mediator of Menthol-induced Analgesia of Acute and Inflammatory Pain. Pain. 154: 2169-2177.
Fernández-Peña, C., and F. Viana. 2013. Targeting TRPM8 for Pain Relief. Open Pain J. 6: 154-164.
He, J., T. L. Pham, A. H. Kakazu, and H. E. P. Bazan. 2019. Remodeling of Substance P Sensory Nerves and Transient Receptor Potential Melastatin 8 (TRPM8) Cold Receptors After Corneal Experimental Surgery. Invest. Ophthalmol. Vis. Sci. 60: 2449-2460.
Lambiase, A., P. Rama, S. Bonini, G. Caprioglio, and L. Aloe. 1998. Topical Treatment with Nerve Growth Factor for Corneal Neurotrophic Ulcers. N. Engl. J. Med. 338: 1174-1180.
Zhang, L., N. Terrando, Z-Z Xu, S. Bang, S-E Jordt, W. Maixner, C. N. Serhan, R-R Ji. 2018. Distinct analgesic action of DHA and DHA-derived specialized pro-resolving mediators on post-operative pain after bone fracture in the mice. Front Pharmacol. 9: 412.
Huang J, J. J. Burston, L. Li, S. Ashraf, P. I. Mapp, A. J. Bennett, S. Ravipati, P. Pousinis, D. A. Barret, B. E. Scammell, V. Chapman. 2017. Targeting the D series resolvin receptor system for the treatment of osteoarthritis pain. Arthritis & Rheumatology 69: 996-1008.
Chen, N., P. Zhou, X. Liu, J. Li, Y. Wan, S. Liu, and F. Wei. 2020. Overexpression of Rictor in the injured spinal cord promotes functional recovery in a rat model of spinal cord injury. FASEB J. Off. Publ. Fed. Am. Soc. Exp. Biol. 34: 6984-6998.

### Example 1

Introduction

Dry eye perturbs vision mainly during aging. It also occurs in rheumatoid arthritis, diabetes, thyroid gland pathologies, environmental conditions (e.g., exposure to smoke or pollutants), long-term use of contact lenses and after refractive surgery. This pathology is triggered by a shortage in tears that lubricate, arrest infections, and nourish and sustain a clear eye surface. Corneal innervation is required to maintain the integrity of the ocular surface (1), and nerve damage decreases tear production, blinking reflex, and perturbs epithelial wound healing, resulting in loss of transparency and vision (2-5). For this reason, there is a strong relationship between dry eye and corneal nerve damage.

Axons from sensory nerves from the ophthalmic branch of the trigeminal ganglion (TG) neurons penetrate the corneal stroma surrounding the limbal area and branch out as the subepithelial plexus before reaching the corneal epithelium, finalizing as free nerve endings (6-8).

After nerve damage occurs from refractive surgeries, (e.g., Laser-assisted in situ keratomileusis, LASIK, or photorefractive keratectomy, PRK), it takes between 3-15 years to recover corneal nerve integrity (9-11). As a consequence, corneal sensitivity decreases and dry-eye disease can develop, causing neuropathic pain, corneal ulcers, and in severe cases, the necessity for corneal transplants (12-14). In addition, dry eye is linked to cold receptor function, mainly the transient receptor potential melastatin 8 (TRPM8) channels (15) that control the corneal surface rate of cooling and maintain normal tear secretion (16-18). In fact, a decrease in TRPM8 terminals takes place, even long after experimental corneal surgery, indicating that these changes contribute to post-surgery neuropathic pain (19).

Topical treatment of the neurotrophin pigment epithelium-derived factor (PEDF) plus the ω-3 fatty acid family member docosahexaenoic acid (DHA) enhances nerve regeneration and stimulates nerve regrowth in rabbit and mouse corneas after experimental surgery, as well as in pathologies like diabetes and herpes virus simplex (HSV1) infection (20-24). Moreover, PEDF activates the Ca²⁺-independent phospholipase A2 (iPLA2ζ) activity of the PEDF receptor (PEDF-R) and releases DHA from membrane phospholipids that can be converted into bioactive docosanoids (25), including neuroprotectin D1 (NPD1) that induces corneal nerve regeneration in a rabbit model of refractive surgery (20). Herein, we report the discovery of a new lipid mediator that is part of the signaling mechanism exerted by PEDF+DHA on the ocular surface. Furthermore, we uncovered that the TG genes sense corneal injury and respond to corneal RvD6si treatment with a specific transcriptomic signature. We demonstrate that the topical application of RvD6si is cornea protective, disclosing a new mechanisms and therapeutic avenues for dry eye and ocular neuropathic pain.

Identification of New Resolvin D6si from Mouse Tears

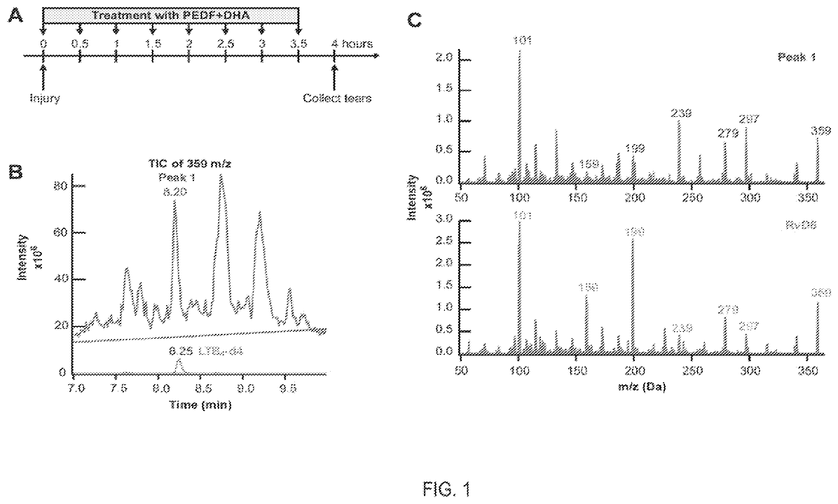

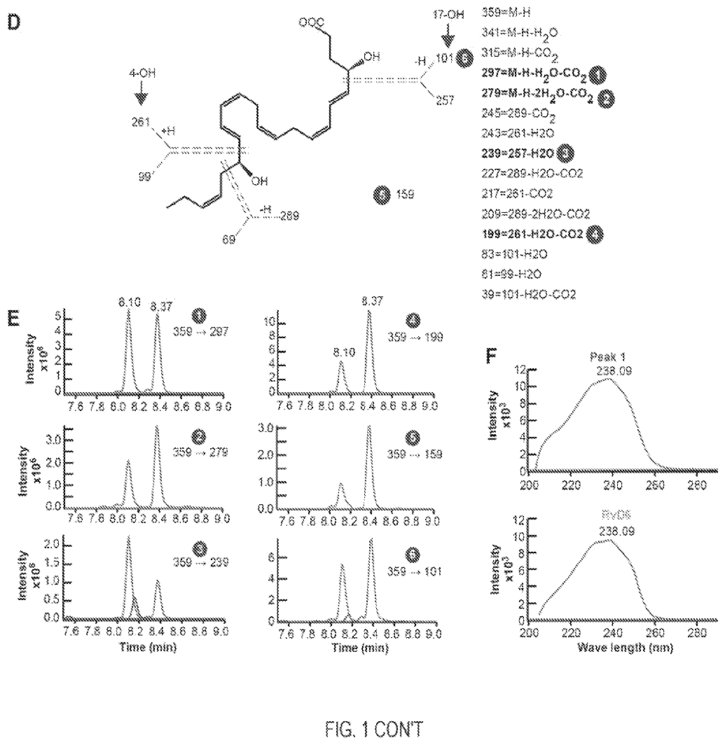

The biological activities of PEDF+DHA have been revealed by our laboratory (20-24). A mechanistic link of PEDF+DHA action on corneal nerve regeneration has been uncovered with the activation of the iPLA2ζ and the increased expression of the neurotrophic factors brain-derived growth factor (BDNF) and nerve growth factor (NGF), and the axon growth guidance semaphorin 7a (Sema7A) released in tears (25). To define which docosanoids are produced after the release of DHA by PEDF activation, mouse corneas were injured and treated, tears collected, and lipids extracted and analyzed by LC-MS/MS (FIG. 1). The total ion chromatogram (TIC) of 359 m/z represented all di-hydroxy DHA isomers in tears after 4 h of treatment, and three peaks were well defined with retention times (RT) 8.20, 8.74, and 9.20 min (FIG. 1). The internal standard LTB4-d4 (green) eluted at 8.25 min. We focused on the peak eluted at 8.20 min (Peak 1) that displays upon full fragmentation a parent ion 359 m/z showing at least 6 matched product ions (daughter ions) compare to the RvD6 standard (FIG. 1) with two hydroxy-groups at Carbon number 4 and 17 (FIG. 1). When Peak 1 was co-injected with RvD6 at the same concentration, Peak 1 eluted 0.27 min earlier than RvD6 at 6 major multiple reaction monitoring (MRM) channels 359->297, 279, 239, 199, 159, and 101 (FIG. 1). The UV spectra for Peak 1 and RvD6 showed maxima absorbance (,max) at 238 nm, revealing that both compounds have conjugated diene structure (FIG. 1). When taken together, our data indicate that Peak 1 is an RvD6 stereospecific isomer (RvD6si) that shares a full fragmentation pattern with RvD6, as well as at least 6 matched daughter ions, 2 hydroxy-groups at C4 and C17 of the DHA backbone and UV spectrum, but has different RT.

RvD6si is Derived from DHA

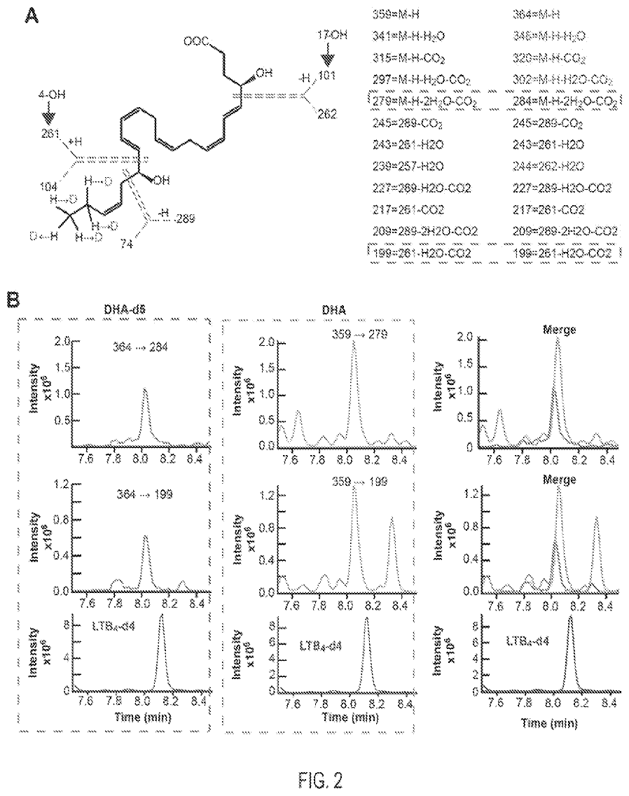

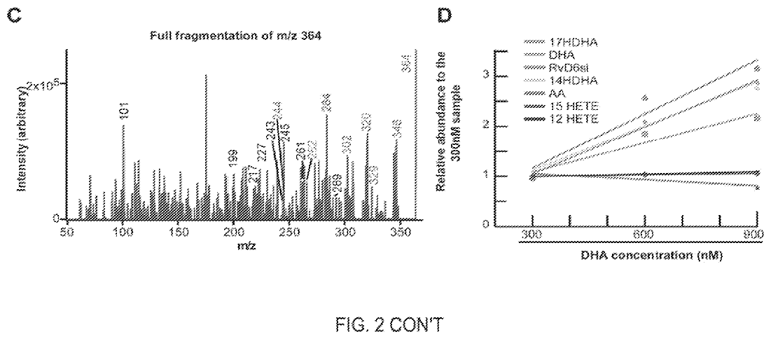

To validate whether the new RvD6si originated from the added DHA, an ex vivo corneal organ culture model was employed (16 corneas/sample). The injured corneas were cultured for 4 h in the presence of DHA or deuterium-labeled DHA (DHA-d5), plus PEDF, and the lipids from the media were extracted and analyzed. Since 5 atoms of deuterium (D) are attached to the end of the DHA backbone (at the 21st and 22nd C), the total mass of RvD6si-d5 was shifted to 365 Da (the [M-H] m/z is 364 in MS results) while some of its product ions were not changed after fragmentation (FIG. 2). The MRM detection method was designed to capture the DHA-d5 total structure. The RvD6si-d5 was detected in the media with a similar RT to the RvD6si produced by PEDF+DHA (FIG. 2). The full fragmentation of RvD6si-d5 confirmed the structure as well (FIG. 2). In addition, the origin of the RvD6si was validated at three different concentrations of added DHA (FIG. 2) with an enhanced synthesis as a function of increased DHA concentration. When analyzing possible arachidonic acid (AA)- and DHA-hydroxy derivatives (HDHA), the results showed a proportional increase of DHA products such as 14- and 17-HDHAs while the amount of AA and its hydroxyl derivatives 12- and 15-HETEs were not changed. These data indicate that the new RvD6si originates from exogenous DHA.

Isolation and Characterization of RvD6si In Vivo

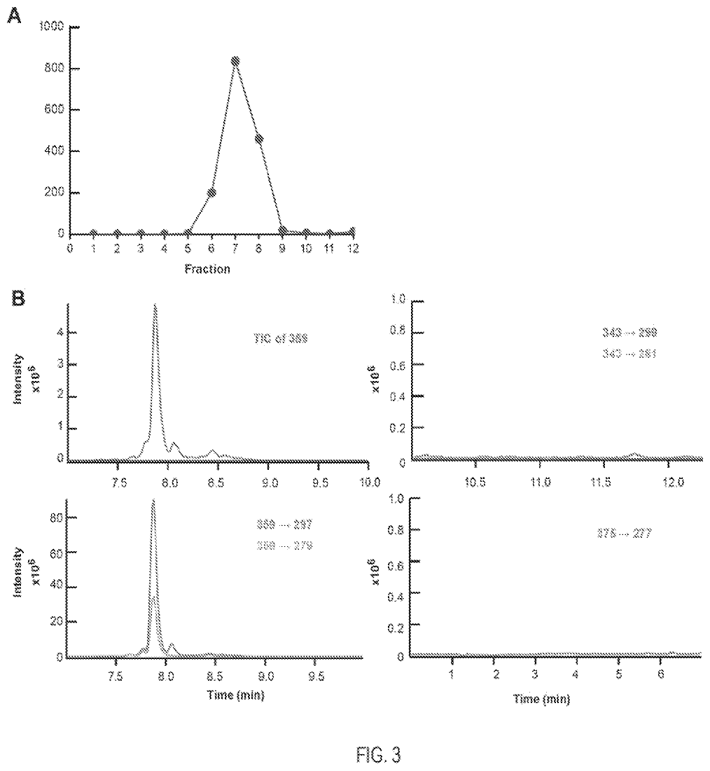

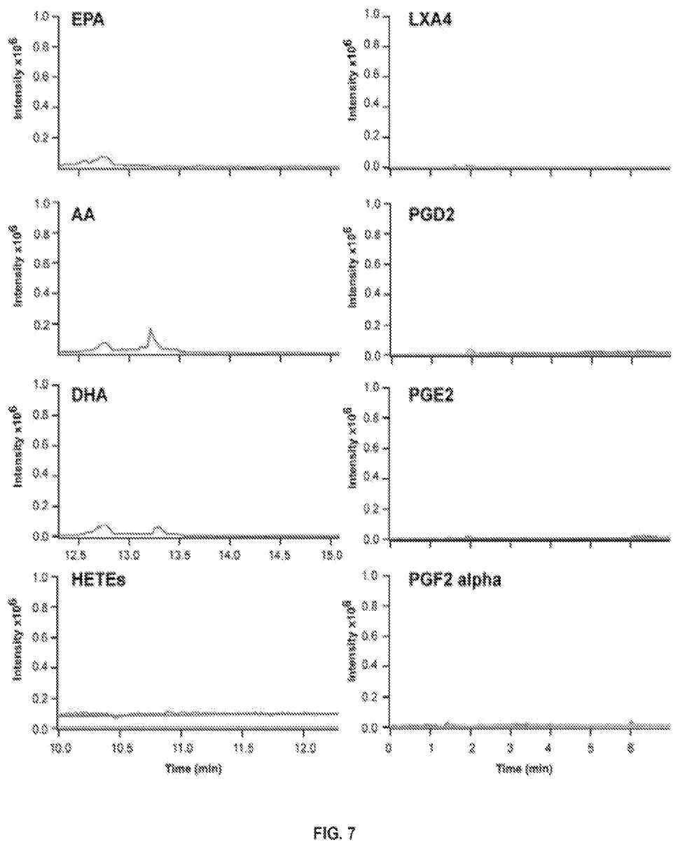

Although the 2D structure of the new RvD6si matched RvD6, the different RT could make them distinct in their biological activities. To obtain enough RvD6si for testing, 60 mice were injured and treated with PEDF+DHA every 30 min for 4 h, and the tears collected. The next day, the mice were euthanized, and the corneas isolated and incubated in media with PEDF+DHA for 4 h. The lipids extracted from tears and corneal media were combined and run in UPLC employing a C18 column, and fractions were collected every 30 sec from 6 to 12 min. All fractions were subject to lipidomic analysis to detect the availability of the new RvD6si. Fractions 6 to 8 with clear detectable amounts of RvD6si were pooled (FIG. 3). The purity of our targeted lipid mediator was determined by lipidomic analysis before being tested in vivo. The isolated RvD6si showed very low contamination of other DHA derivatives (FIG. 3) as well as AA, eicosapentaenoic acid (EPA), and their derivatives (FIG. 7).

RvD6si Enhances Corneal Wound Healing and Recovery of Corneal Sensitivity after Injury

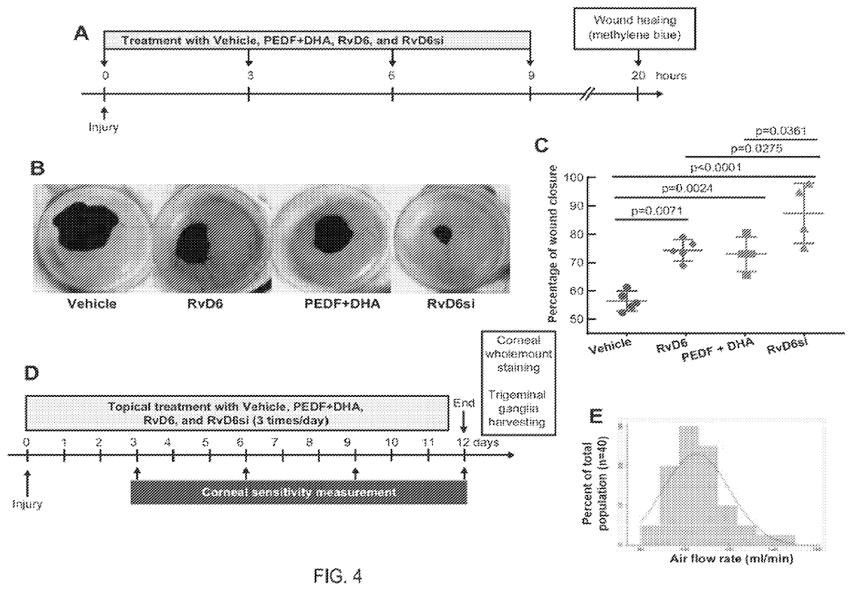

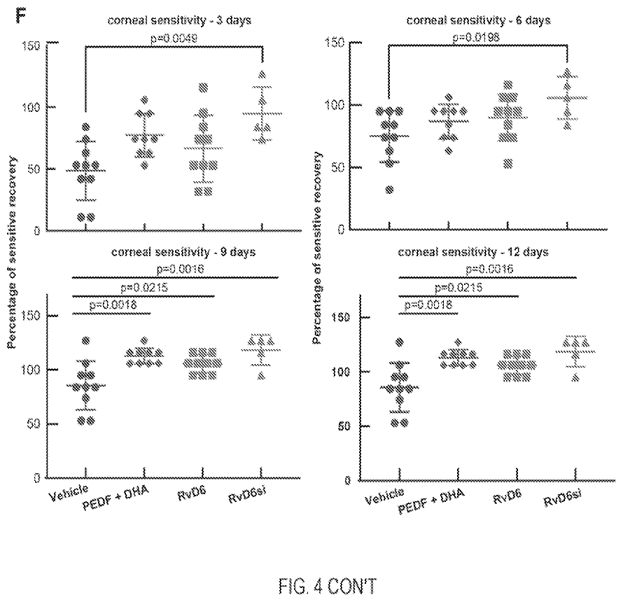

Studies have shown that PEDF+DHA promotes corneal wound healing in rabbit (20, 21), and in normal and diabetic mice (24, 25) after experimental surgery. We validated the ability of RvD6s (either RvD6 or RvD6si) in stimulating corneal wound healing. The right mouse eyes were injured, and the animals were divided into four groups: vehicle, PEDF+DHA, RvD6, and RvD6si (FIG. 4). Twenty hours after injury, all drug-treated mice had faster corneal wound healing than vehicle; however, the greatest increase was found in the animals treated with the RvD6si (FIG. 4).

Corneal sensitivity was evaluated at days 3, 6, 9 and 12 after corneal injury and treatment (FIG. 4). A new methodology to measure the sensation in mouse cornea was introduced using the Belmonte non-contact aesthesiometer. FIG. 4 shows a Gaussian-curve of distribution from basal corneal sensation recorded-values (n=40 corneas) at a flow rate of 100.45 to 110.05 ml of air/minute (α=0.05). It is important to note that this range of normal corneal sensitivity is critical to evaluate corneal sensation since the Belmonte non-contact aesthesiometer working flow rate is from 20 to 200 ml/min. The range of normal corneal sensitivity from 100.45 to 110.05 ml in the mouse was regarded as successful recovery after injury and treatment, and it was used to normalize the measurements. There was a faster recovery of corneal sensation in the animals treated with the RvD6si at 3 and 6 days after injury (FIG. 4). By 9 days, the three treatments increased the sensitivity compared to vehicle, and at 12 days, there was no significant difference in any of the studied groups.

RvD6si Enhances Corneal Nerve Regeneration.

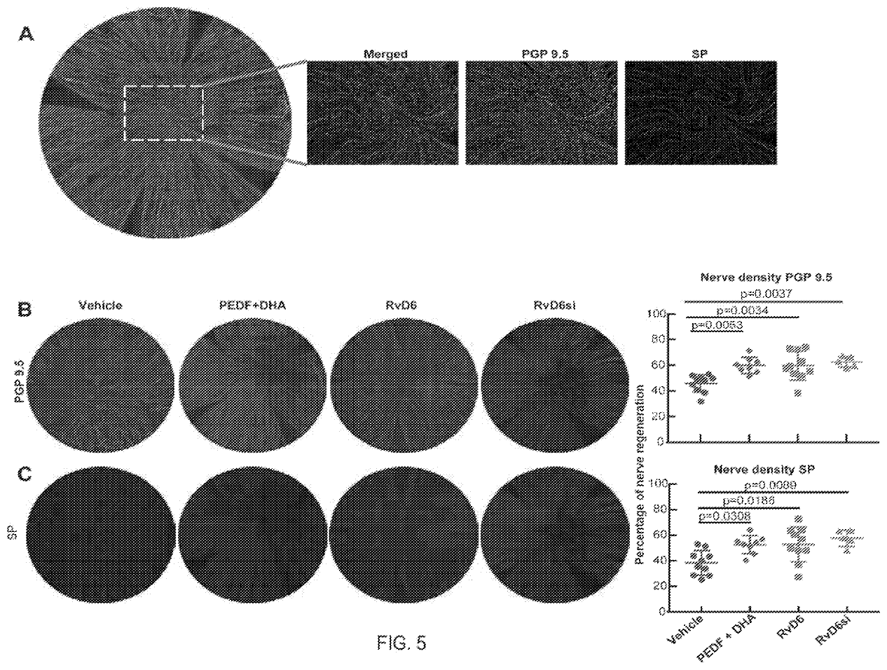

PEDF+DHA stimulates corneal nerve regeneration in injury animal models (20-25). It was important to confirm the biological activity of RvD6si as a lipid mediator underlying the action of PEDF+DHA. To validate this, mice were injured and treated (as described in FIG. 4). Isolated corneas were stained with PGP 9.5, a pan-neuronal marker, and with SP neuropeptide antibodies. The density of non-injured corneal nerves positive to PGP 9.5 and SP, respectively, was used to normalize the values (FIG. 5). Substance P is a main neuropeptide in mammalian corneas (26-28). Moreover, a previous study from our group has demonstrated that there is a correlation between corneal sensitivity and SP-positive nerves (29).

At 12 days after injury and treatment, total corneal nerve density was 45.9±6.8% of the normal cornea in the vehicle-treated group and significantly higher in the RvD6si treated corneas 62.6±4.2% (p<0.05) (FIG. 5). PEDF+DHA and RvD6 treatment also increased nerve density to 59.9±63% and 59.7±11.2%, respectively. There were no significant differences between RvD6si, RvD6, and PEDF+DHA. Similarly, the density of SP-positive nerves at 12 days after injury was higher with RvD6si, RvD6 and PEDF+DHA treatments compared to the vehicle-treated group (FIG. 5). This result confirmed a faster recovery of corneal sensitivity in treated corneas (FIG. 4) and strengthened the biological function of the RvD6si as the main mediator in the mechanism of PEDF+DHA to enhance corneal nerve regeneration.

Transcriptome Selective Modulation by RvD6si in the Trigeminal Ganglion

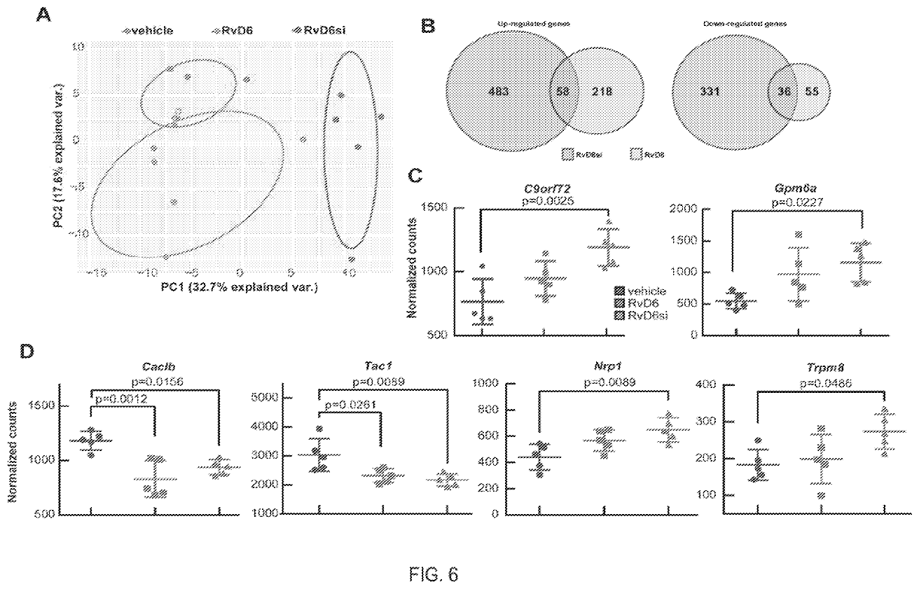

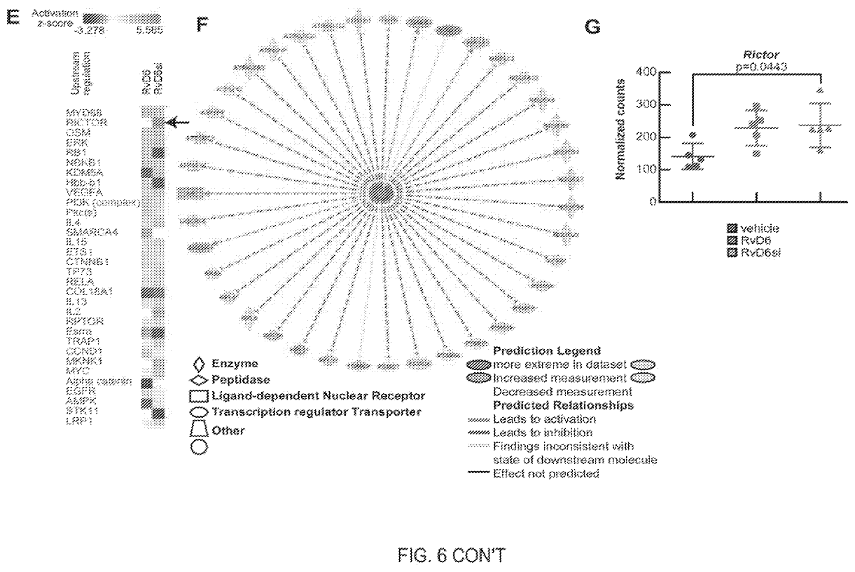

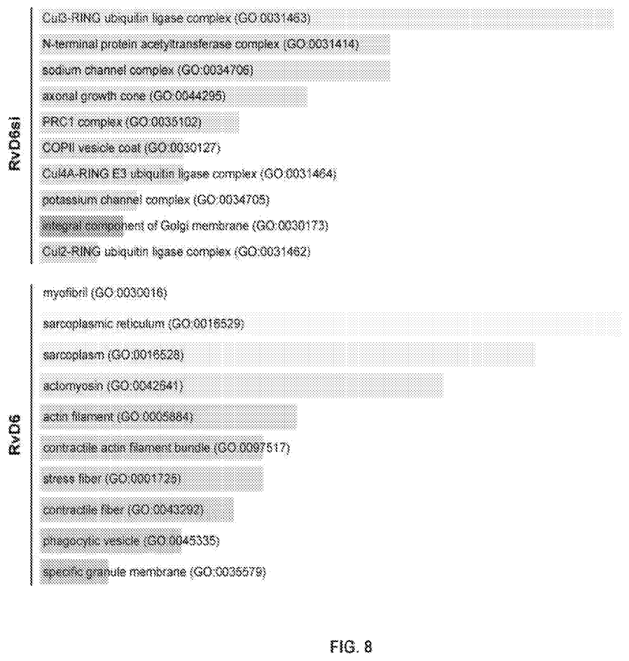

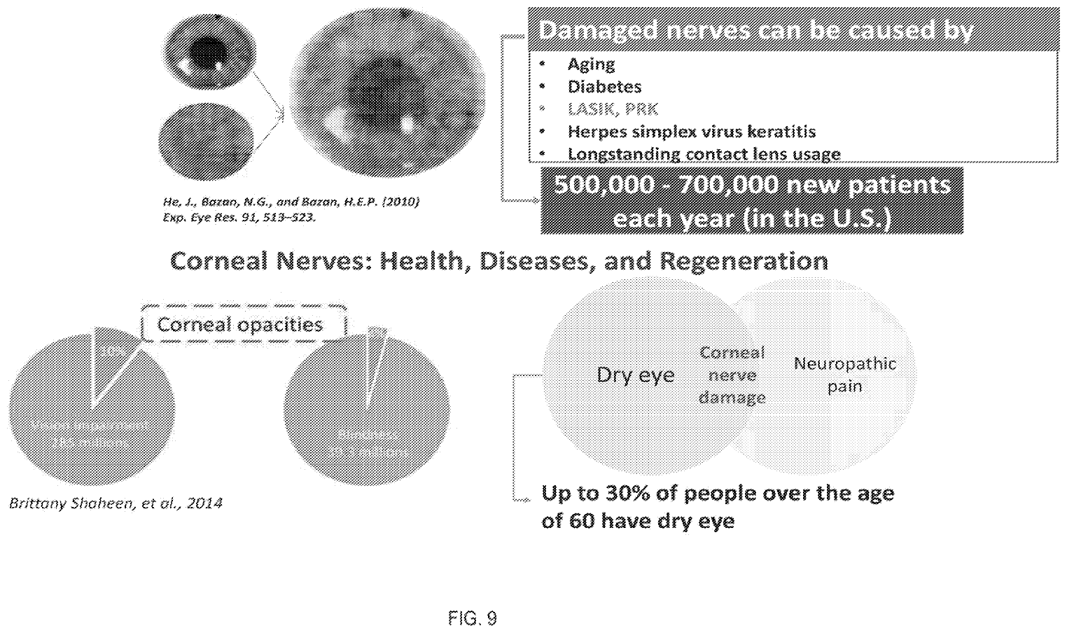

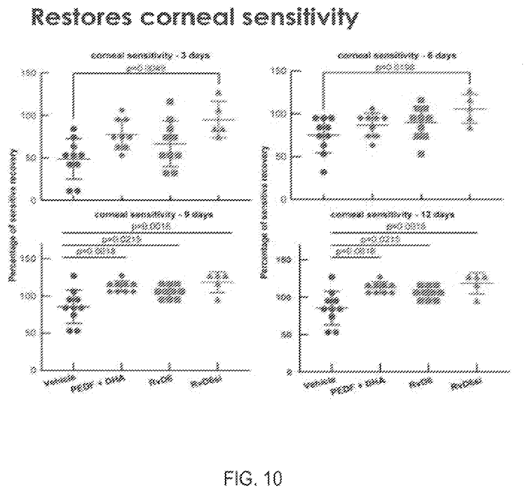

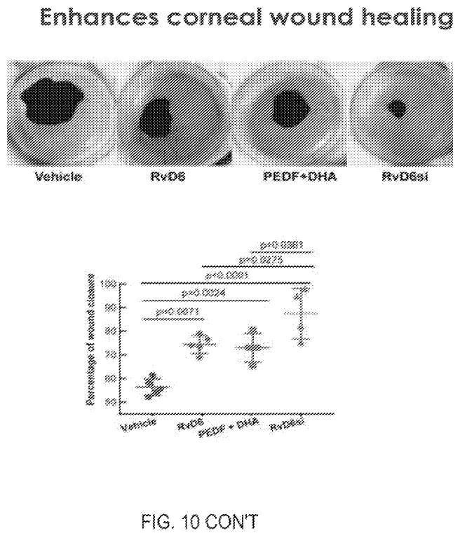

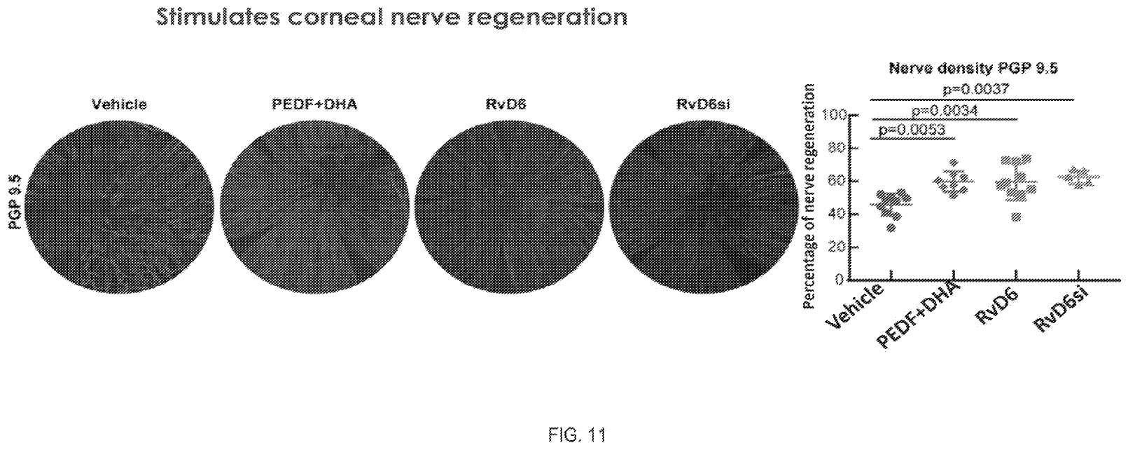

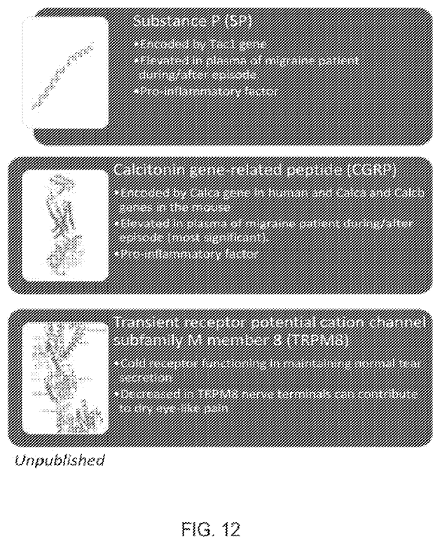

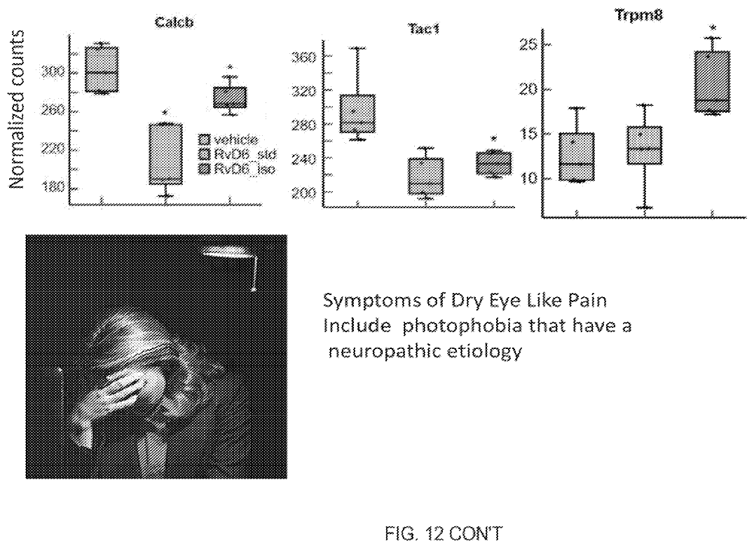

Because corneal sensory nerves originate in TG neurons, we wanted to validate whether corneal injury could be sensed in the TG and t, in turn, would elicit a gene expression response. Thus, TG were harvested 12 days after injury and treatment with RvD6si or RvD6 or vehicle treatment used as control (FIG. 4) and then RNA-seq analysis was performed. Quality controls showed that mapped reads range from 84.63 to 93.00%, with about 20,000 expressed genes/sample. Principal component analysis (PCA) showed good separation of vehicle-treated from the RvD6si- or RvD6-treated groups (FIG. 6). The two RvD6s shared 58 upregulated genes and 36 downregulated genes compared to controls (FIG. 6). To classify upregulated genes of RvD6si_vs_vehicle and RvD6_vs_vehicle, gene enrichment analysis was used to demonstrate that the RvD6si showed a difference in cellular comparted locations (FIG. 8) and that activate axonal growth cone genes (gene ontology number 0044295). The box plots depict two activated genes by RvD6si in this class: C9orf72 and Gpm6a (FIG. 6). We also detected specific genes related to neuropeptides and ion channel receptors in the cornea that are stimulated by the addition of PEDF+DHA (19, 21, 24) (FIG. 6D). The RNA-seq established that RvD6 or RvD6si reduced gene expression of two major neuropeptides, tachykinin precursor 1 (Tac1) that encodes Substance P (SP) and calcitonin-related polypeptide beta (Calcb). It is important to note that these neuropeptides, especially Calcb, are major pain-induced mediators in migraine and other primary headaches (30). In contrast, the RvD6si selectively enhanced the expression of transient receptor potential melastatin 8 (Trpm8) channel, and neuropilin 1 (Nrp1), the co-receptor for several factors including class III/IV semaphorins, certain isoforms of vascular endothelial growth factor, and transforming growth factor beta (31).

Further analysis revealed a strong induction by RvD6si of the transcriptional factor Rictor (FIG. 6) that is a part of the rapamycin-insensitive mammalian target complex-2 (mTORC2) (FIG. 6). There were 39 genes modulated by RICTOR modified by RvD6si. Among those, 37 (95%) genes matched the IPA knowledge collected from published data, while only two genes, Egr1 and Psme3, did not fit with the prediction (yellow arrows) (FIG. 6). It is important to note that all genes subjected to IPA analysis are significantly different (FDR<0.05 in the DESeq2 analysis) in comparison to vehicle-treated group. For this reason, 95% of downstream genes matched to IPA knowledge; the Rictor signaling is clearly stimulated in TG by RvD6si (FIG. 6).

Discussion

Studies from our laboratory have demonstrated the use of PEDF+DHA for corneal wound healing and nerve regeneration in post-surgical models of rabbits and mice (20-25). This included the observation that activation of the iPLA2ζ activity of the PEDF-R releases DHA from phospholipids, suggesting that docosanoids could be synthesized in the cornea (25). Here, we report the finding, identification and characterization of its bioactivity of a new Resolvin D6si in tears that is derived from DHA upon activation of PEDF on its receptor. The full MS/MS fragmentation of the RvD6si matches six characteristic ions with the RvD6 as well as the UV diode array profile (FIG. 1). The biological activity revealed that it enhances corneal wound healing and sensitivity recovery, more potently than PEDF+DHA after corneal PRK-mimic surgery (FIG. 4). These results indicate that RvD6si is the main lipid mediator that contributes to the signaling mechanism of the action of PEDF+DHA. Moreover, the RvD6s and PEDF+DHA treatments show similar enhancement in corneal innervation at 12 days after injury and treatment (FIG. 6).

Resolvin D6 was described using human polymorphonuclear neutrophils (32) and was detected in skin (33), brain (34), cerebrospinal fluid (35), and plasma (36). However, this is the first report demonstrating a biological function of RvD6 and of a novel stereoisomer. The formation of potent bioactive mediators from DHA was proposed when mono-, di-, and tri-hydroxy DHA-derivatives were detected as enzyme-mediated products of oxygenated metabolites of DHA in the retina (37). Unlike the retina, where photoreceptor membranes have high DHA content esterified at the sn-2 position of phospholipids (38), the cornea contains more AA at that position (25, 39). For this reason, the addition of exogenous DHA is required to synthesize docosanoids rather than eicosanoids. Further, the RvD6si was not detected when corneas were treated with DHA or PEDF alone, indicating that new RvD6si is only detected when corneas are treated with PEDF+DHA. This observation is in agreement with a previous study showing that neither RvD6 nor its stereoisomers were detected in human tear samples (40). Since the RvD6si was found primarily in the tears or media of corneas in organ culture, this indicates that the RvD6si needs to be secreted into the extracellular compartment to become functional. The biological activity can be elicited through a receptor and, in turn, modulates cell signaling and transcription factors, upregulating, as a consequence, neurotrophic genes in the cornea (25). RvD6si can act in autocrine fashion and/or may diffuse through tears and act as a paracrine signal on other ocular surface cells.

Most of the corneal nerves originate from neurons localized in the TG (6). Therefore, using unbiased RNA sequencing, we have deciphered here that RVD6 and RvD6si shared a small number of unregulated genes in the TG, implicating that the signaling mechanism of their biological activities have differences. The RNA-seq data reveal a strong activation by the RvD6si of two genes, C9orf72 and Gpm6A, that stimulate neurogenesis and growth cone formation (41, 42). We also found genes related to pain since corneal neuropathic pain can occur after nerve damage (43). The expression of two genes involved in pain was decreased in corneas treated with the RvD6si: Tac1 that encodes SP, which is one of the most abundant neuropeptides expressed in corneal nerves (26-28). SP exerts proinflammatory effects, and preclinical studies linked their action to chronic pain (44). The other is Calcb, which encodes Calcitonin gene-related peptide (CGRP), which is also abundant in corneal nerves (21) and plays an essential role in neurogenic inflammation and pain (30). Another important gene in this category is Trmp8. TRPM8 channels regulate the wetting of the ocular surface and have an analgesic effect on chronic pain (17, 46-49). Previous studies in mice where the nerves had been damaged showed that TRPM8-positive nerve fibers only reach 50% of their normal density by 3 months after the injury, indicating that the decrease in TRPM8 nerve terminals can contribute to dry eye-like pain (19). The increased expression of Trpm8 after injury and treatment with RvD6si indicates that the new lipid could protect corneas from pain. In addition, the selective increase of Nrp1 is also interesting, since it is the co-receptor of SEMA3A that has been shown to attenuate mechanical allodynia in a rat model of sciatic nerve injury (50).

Our results disclose that the RvD6si potently and selectively induces Rictor gene expression in the TG. As a regulator of PI3K/Akt pathways, RICTOR is a key component of mTORC2 and is clearly involved in cell proliferation and repair. In agreement with this, the deletion of Rictor or mTORC2 inhibited the sensory-axonal regeneration in mice after dorsal root ganglion injury (51).

In conclusion, our data demonstrate that a new RvD6si produced by the injured cornea after PEDF+DHA treatment is necessary for corneal wound healing and nerve regeneration. This lipid mediator activates signaling that communicates from the cornea to TG neurons, and as a response, modulates specific gene signatures that enhance axon growth, decrease neuropathic pain and foster containment of dry eye. Our findings provide compositions and methods using RvD6si for impaired-corneal nerve diseases, including dry eye, corneal neurotrophic ulcers, neurotrophic keratitis and neuropathic pain.

Animals

Ten-week-old male CD1 mice were purchased from Charles River (Wilmington, Mass., USA) and maintained in a 12-h dark/light cycle at 30 lux at the animal care facility at the Neuroscience Center of Excellence, Louisiana State University Health New Orleans, New Orleans, La. The animals were handled in compliance with the guidelines of the Association for Research in Vision and Ophthalmology Statement for the Use of Animals in Ophthalmic and Vision Research, and the experimental protocols were approved by the Institutional Animal Care and Use Committee at Louisiana State University Health New Orleans.

Corneal Injury and Treatment

Mice were anesthetized with a mix of ketamine (200 mg/kg) and xylazine (10 mg/kg) injected intraperitoneally, and one drop of proparacaine hydrochloride solution (0.5%) was applied to the right eye subjected to injury. As previously described (19, 29), the center of the cornea was demarcated with a 2 mm trephine, and the epithelium and the anterior stroma were gently removed under a surgical microscope using a corneal rust ring remover (Algerbrush II; Alger Equipment Co., Lago Vista, Tex., USA). One drop of 0.3% of tobramycin ophthalmic solution (Henry Schein, Melville, N.Y., USA) was applied to the eye to prevent postoperative infection. The same investigator (J. H.) performed all surgeries. Afterward, 10 μl of PEDF (50 ng/ml) plus DHA (50 nM) or DHA-derived lipid mediators were applied topically, as explained in each experimental design.

Lipidomic Analysis

Five microliters of sterile PBS was instilled in the inferior cul-de-sac of the mouse eye, and 30 s later, tears were collected in 1 mL of ice-cold MeOH containing 1 g/L Butylated hydroxytoluene followed by the addition of 2 ml of CHCl₃ and 5 μl of an internal standard mixture of deuterium-labeled lipids AA-d8 (5 ng/μl), PGD2-d4 (1 ng/μl), EPA-d5 (1 ng/μl), 15-HETE-d8 (1 ng/μl), and LTB4-d4 (1 ng/μl). The samples were sonicated in a water bath for 30 min and stored at −80° C. overnight. The next day, the samples were centrifuged, supernatant was collected, and the pellet was washed with 1 ml of CHCl₃/MeOH (2:1) and centrifuged, and then the supernatants were combined. Water, pH 3.5, was added to the supernatant at the ratio 1:5, vortexed, and centrifuged, the pH of the upper phase was adjusted to 3.5-4.0 with 1 N HCl. The lower phase was collected, dried under N₂ and then resuspended in 1 ml of MeOH and stored at −80° C.

For corneal organ culture experiments, 2 mL of media was collected and centrifugated at 14,000 rpm for 15 min at 4° C. to remove cellular debris. Lipids were extracted by the Blight and Dyer method (52). Briefly, 3.75 ml of a mixture of CHCl₃: MeOH (1:2) was added to 1 ml of sample and 5 μl of the deuterium-labeled internal standard mixture of lipids. The samples were vortexed and stored at −80° C. overnight. Next, to make two phases, 2.5 ml of CHCl₃ was added and vortexed, and then 2.5 mL of water (pH 3.5) was added, vortexed, and the pH of the upper phase adjusted to 3.5-4.0 with 1 N HCl. The lower phase was dried down under N₂, resuspended in 1 ml of MeOH, and stored at −80° C.

LC-MS/MS analysis was performed in a Xevo TQ equipped with Acquity I class ultra-performance liquid chromatography (UPLC) with a flow-through needle (Waters Corporation, Milford, Mass.). As described (25, 53), samples were dried under N₂, resuspended in 20 μl of MeOH/H₂O (2:1), and injected into a CORTECS C18 2.7 μm 4.6×100 mm column (Water, Mass.). The column temperature was set at 45° C. with a flow of 0.6 ml/min. The initial mobile phase consisted of 45% solvent A (H₂O+0.01% acetic acid) and 55% solvent B (MeOH+0.01% acetic acid) and then a gradient to 15% solvent A for the first 10 min followed by a gradient to 2% solvent A for 18 min, 2% solvent A run isocratically until 25 min, and then a gradient back to 45% solvent A for re-equilibration until 30 min. Lipid standards (Cayman, Ann Arbor, Mich.) were used for tuning and optimization, as well as to create calibration curves for each compound. RvD6 [4S,17S-dihydroxy-5E,7Z,10Z,13Z,15E,19Z-docosahexaenoic acid] standard was provided

Production of Resolvin D6si from Mouse Tears and Cornea

Mouse corneas (n=60) were injured and treated topically with PEDF+DHA for 4 h. Tears were collected in MeOH and stored at −80° C. After 24 h, mice were euthanized, and injured corneas were excised and cultured with PEDF+DHA in DMEM/F12 media for 4 h. The medium was collected, and lipids were extracted as described above. Lipids from pooled tears and cornea-cultured media were subjected to UPLC separation using a C18 column (Water, Mass.). Twelve fractions (30 sec/fraction) between 6-12 min after injection were collected. The procedure was repeated at least 8 times with 25 μl of sample/run until all the sample was fractionated. Each fraction was dried under N₂ and resuspended in 1 mL of MeOH. The presence of RvD6si in 10 μl of each fraction was confirm using the described LC-MS/MS system. The fractions with high purity and concentration of RvD6si were pooled and stored at −80° C. until needed for the in vivo experiments.

Corneal Wound Healing

Mice were euthanized 20 h after injury and treatment, and corneas were stained with 0.5% methylene blue for 20 sec and then washed with PBS for 2 min. Photographs were taken with a dissecting microscope (SMZ 1500; Nikon, Tokyo, Japan) through an attached digital camera (DXM 1200; Nikon). The images corresponding to the wounded area were quantified using Photoshop CC 2014 software (Adobe, San Jose, Calif., USA).

Corneal Sensitivity Measurement

The non-contact corneal aesthesiometer has been described as a more reliable method than the standard Cochet-Bonnet aesthesiometer to determine the corneal sensation threshold (54). Therefore, for corneal sensation measurement, the Belmonte non-contact corneal aesthesiometer (55) was used with some modification. Briefly, one researcher held the mouse and kept the air output needle at a distance of 3 mm from the cornea. Another researcher controlled the air flow rate. The measurements started at an air flow rate of 80 ml per minute and then increased gradually by ten units until the mouse started blinking. When the mouse blinked, the air flow rate was recorded as the final corneal sensitivity index.

Corneal Nerve Analysis

Twelve days after injury and treatment, mice were euthanized, and the eyes enucleated and fixed with Zamboni's fixative (American Master Tech Scientific, Lodi, Calif., USA) for 45 min at room temperature. The corneas were then excised and fixed for an additional 15 min, followed by 3 washes with PBS. To block nonspecific binding, corneas were incubated with 10% normal goat serum plus 0.5% Triton X-100 in PBS for 1 h at room temperature. Afterward, corneas were incubated with the primary antibodies, rabbit monoclonal anti-PGP9.5 (1:500), (ab108986; Abcam, Cambridge, Mass., USA), and rat monoclonal anti-substance-P (SP; 1:100) (sc-21715; Santa Cruz Biotechnology, Dallas, Tex., USA) for 24 h at room temperature with constant shaking. After being washed with PBS, the corneas were incubated with the corresponding secondary antibodies goat anti-rabbit Alexa-Fluor 488 (1:1000) and goat anti-rat Alexa-Fluor 488 (1:1000) (Thermo Fisher Scientific, Waltham, Mass., USA) for 24 h at 4° C. Four radial cuts were performed on each cornea that was flatly mounted on a slide with the endothelium side up and examined with a fluorescent microscope (Deconvolution microscope DP80; Olympus, Tokyo, Japan). The images were merged together to build the entire view of the corneal nerve network. The corneal nerve density was measured using Photoshop CC 2014 (Adobe) as previously described (26, 29).

Trigeminal Ganglion RNA Sequencing

TG corresponding to the injury eye side (n=5) were harvested and kept in RNAlater solution (Thermo Fisher Scientific) until homogenized on ice using a Dounce homogenizer. Total mRNA was extracted using an RNeasy mini kit (Qiagen, Germantown, Md., USA) as described by the manufacturer. Purity and concentration of RNA were determined with a NanoDrop ND-1000 spectrophotometer (Thermo Fisher Scientific), and the samples were stored at −80° C. until used. RNA sequencing was performed using the adapted Smart-seq2 protocol (56). Briefly, one ng of total RNA was reverse transcribed with the Oligo-dT30VN and template-switching oligo (TSO) primers. The total cDNAs were amplified using ISPCR primer, and the library was made using the Nextera XT DNA library preparation kit (Illumina, San Diego, Calif., USA). The libraries were pooled using the same molarity and sequenced using the NextSeq 500/550 High Output Kit v2 (75 cycles, Illumina). After demultiplexing, RNA-seq data were aligned to the GENCODE GRCm38 mouse primary genome assembly (Release M22, gencodegenes.org/mouse/) using the RSubread package v1.34.6 for R v3.6.1 (57). The outputted BAM files for sequencing data alignment were counted using featureCounts function (Subread v1.6.5 in Ubuntu LTS 16.4 operating system) (58). Next, the raw count data were subjected to differential gene expression analysis using DESeq2 package for R (59). The adjusted p-values were regarded as the false discover rate (FDR). Significantly changed genes (FDR<0.05) between RvD6si_vs_vehicle and RvD6_vs_vehicle were subjected to the enrichment analysis using Enrichr (60) and pathway analysis using the IPA (QIAGEN Inc., https://www.qiagenbioinformatics.com/products/ingenuity-pathway-analysis).

Statistical Analysis

Data are expressed as mean±SD of ≥3 independent experiments. The data was analyzed by 1-way ANOVA followed by Tukey honest significant difference post hoc test at 95% confidence level to compare the different groups and considered significant when p<0.05. All statistical analyses were performed using the Stata 14 (StataCorp, College Station, Tex., USA). Graphs were made using Prism 7 software (GraphPad Software, La Jolla, Calif., USA) and Bio Vinci (BioTuring, La Jolla, Calif., USA). For the sequencing data, since the DE-Seq2 analysis does not provide the multi-samples comparison, the normalized counts from DE-Seq2 were used as the input of ANOVA test.

Accession Numbers

Completed RNA-Seq data that support the findings of this study have been deposited in Gene Expression Omnibus with the accession code GSE138685.

### REFERENCES CITED IN THIS EXAMPLE 1

1. Shaheen B S, Bakir M, Jain S. Corneal nerves in health and disease. Surv Ophthalmol 2014; 59(3):263-285.
2. He J, Bazan H E P. Mapping the nerve architecture of diabetic human corneas. Ophthalmology 2012; 119(5):956-964.
3. Hamrah P et al. Corneal sensation and subbasal nerve alterations in patients with herpes simplex keratitis: an in vivo confocal microscopy study. Ophthalmology 2010; 117(10):1930-1936.
4. Cruzat A et al. Inflammation and the nervous system: the connection in the cornea in patients with infectious keratitis. Invest. Ophthalmol. Vis. Sci. 2011; 52(8):5136-5143.
5. He J, Bazan H E P. Corneal nerve architecture in a donor with unilateral epithelial basement membrane dystrophy. Ophthalmic Res 2013; 49(4):185-191.
6. Müller L J, Marfurt C F, Kruse F, Tervo T M T. Corneal nerves: structure, contents and function. Exp. Eye Res. 2003; 76(5):521-542.
7. He J, Bazan N G, Bazan H E P. Mapping the entire human corneal nerve architecture. Exp. Eye Res. 2010; 91(4):513-523.
8. Patel D V, McGhee C N J. Mapping of the normal human corneal sub-Basal nerve plexus by in vivo laser scanning confocal microscopy. Invest. Ophthalmol. Vis. Sci. 2005; 46(12):4485-4488.
9. Erie J C, McLaren J W, Hodge D O, Bourne W M. Recovery of corneal subbasal nerve density after PRK and LASIK. Am. J. Ophthalmol. 2005; 140(6):1059-1064.
10. Chao C, Golebiowski B, Stapleton F. The role of corneal innervation in LASIK-induced neuropathic dry eye. Ocul Surf 2014; 12(1):32-45.
11. Kymionis G D et al. Fifteen-year follow-up after anterior chamber phakic intraocular lens implantation in one and LASIK in the fellow eye. Semin Ophthalmol 2009; 24(6):231-233.
12. Linna T U et al. Effect of myopic LASIK on corneal sensitivity and morphology of subbasal nerves. Invest. Ophthalmol. Vis. Sci. 2000; 41(2):393-397.
13. Lee B H, McLaren J W, Erie J C, Hodge D O, Bourne W M. Reinnervation in the cornea after LASIK. Invest. Ophthalmol. Vis. Sci. 2002; 43(12):3660-3664.
14. Hovanesian J A, Shah S S, Maloney R K. Symptoms of dry eye and recurrent erosion syndrome after refractive surgery. J Cataract Refract Surg 2001; 27(4):577-584.
15. Rosenthal P, Borsook D. Ocular neuropathic pain. British Journal of Ophthalmology 2016; 100(1):128-134.
16. Hirata H, Meng I D. Cold-Sensitive Corneal Afferents Respond to a Variety of Ocular Stimuli Central to Tear Production: Implications for Dry Eye Disease. Invest. Ophthalmol. Vis. Sci. 2010; 51(8):3969-3976.
17. Belmonte C, Gallar J. Cold Thermoreceptors, Unexpected Players in Tear Production and Ocular Dryness Sensations. Invest. Ophthalmol. Vis. Sci. 2011; 52(6):3888-3892.
18. Robbins A, Kurose M, Winterson B J, Meng I D. Menthol Activation of Corneal Cool Cells Induces TRPM8-Mediated Lacrimation but Not Nociceptive Responses in Rodents. Invest. Ophthalmol. Vis. Sci. 2012; 53(11):7034-7042.
19. He J, Pham T L, Kakazu A H, Bazan H E P. Remodeling of Substance P Sensory Nerves and Transient Receptor Potential Melastatin 8 (TRPM8) Cold Receptors After Corneal Experimental Surgery. Invest. Ophthalmol. Vis. Sci. 2019; 60(7):2449-2460.
20. Cortina M S, He J, Li N, Bazan N G, Bazan H E P. Neuroprotectin D1 Synthesis and Corneal Nerve Regeneration after Experimental Surgery and Treatment with PEDF plus DHA. Invest Ophthalmol Vis Sci 2010; 51(2):804-810.
21. Cortina M S, He J, Li N, Bazan N G, Bazan H E P. Recovery of corneal sensitivity, calcitonin gene-related peptide-positive nerves, and increased wound healing induced by pigment epithelial-derived factor plus docosahexaenoic acid after experimental surgery. Arch. Ophthalmol. 2012; 130(1):76-83.
22. He J, Cortina M S, Kakazu A, Bazan H E P. The PEDF Neuroprotective Domain Plus DHA Induces Corneal Nerve Regeneration After Experimental Surgery. Invest. Ophthalmol. Vis. Sci. 2015; 56(6):3505-3513.
23. He J et al. PEDF plus DHA modulate inflammation and stimulate nerve regeneration after HSV-1 infection. Exp. Eye Res. 2017; 161:153-162.
24. He J, Pham T L, Kakazu A, Bazan H E P. Recovery of Corneal Sensitivity and Increase in Nerve Density and Wound Healing in Diabetic Mice After PEDF Plus DHA Treatment. Diabetes 2017; 66(9):2511-2520.
25. Pham T L et al. Defining a mechanistic link between pigment epithelium-derived factor, docosahexaenoic acid, and corneal nerve regeneration. J. Biol. Chem. 2017; 292(45):18486-18499.
26. He J, Bazan H E P. Neuroanatomy and Neurochemistry of Mouse Cornea. Invest. Ophthalmol. Vis. Sci. 2016; 57(2):664-674.
27. Tervo K et al. Substance P-immunoreactive nerves in the human cornea and iris. Invest. Ophthalmol. Vis. Sci. 1982; 23(5):671-674.
28. He J, Pham T L, Bazan H E P. Mapping the entire nerve architecture of the cat cornea. Vet Ophthalmol 2019; 22(3):345-352.
29. Pham T L, Kakazu A, He J, Bazan H E P. Mouse strains and sexual divergence in corneal innervation and nerve regeneration. FASEB J. 2018; fj201801957R.
30. Iyengar S, Ossipov M H, Johnson K W. The role of calcitonin gene-related peptide in peripheral and central pain mechanisms including migraine. Pain 2017; 158(4):543-559.
31. Chaudhary B, Khaled Y S, Ammori B J, Elkord E. Neuropilin 1: function and therapeutic potential in cancer. Cancer Immunol. Immunother. 2014; 63(2):81-99.
32. Serhan C N et al. Resolvins: a family of bioactive products of omega-3 fatty acid transformation circuits initiated by aspirin treatment that counter proinflammation signals. J. Exp. Med. 2002; 196(8):1025-1037.
33. Motwani M P et al. Pro-resolving mediators promote resolution in a human skin model of UV-killed Escherichia coli-driven acute inflammation. JCI Insight 2018; 3(6). doi:10.1172/jci.insight.94463
34. Marcheselli V L et al. Novel docosanoids inhibit brain ischemia-reperfusion-mediated leukocyte infiltration and pro-inflammatory gene expression. J. Biol. Chem. 2003; 278(44):43807-43817.
35. Mai N T et al. A randomised double blind placebo controlled phase 2 trial of adjunctive aspirin for tuberculous meningitis in HIV-uninfected adults. Elife 2018; 7. doi:10.7554/eLife.33478
36. Elajami T K et al. Specialized proresolving lipid mediators in patients with coronary artery disease and their potential for clot remodeling. FASEB J. 2016; 30(8):2792-2801.
37. Bazan N G, Birkle D L, Reddy T S. Docosahexaenoic acid (22:6, n-3) is metabolized to lipoxygenase reaction products in the retina. Biochem. Biophys. Res. Commun. 1984; 125(2):741-747.
38. Anderson R E, Maude M B. Lipids of ocular tissues: VIII. The effects of essential fatty acid deficiency on the phospholipids of the photoreceptor membranes of rat retina. Archives of Biochemistry and Biophysics 1972; 151(1):270-276.
39. Bazan H E, Bazan N G. Composition of phospholipids and free fatty acids and incorporation of labeled arachidonic acid in rabbit cornea. Comparison of epithelium, stroma and endothelium. Curr. Eye Res. 1984; 3(11):1313-1319.
40. English J T, Norris P C, Hodges R R, Dartt D A, Serhan C N. Identification and Profiling of Specialized Pro-Resolving Mediators in Human Tears by Lipid Mediator Metabolomics. Prostaglandins Leukot Essent Fatty Acids 2017; 117:17-27.
41. Sivadasan R et al. C9ORF72 interaction with cofilin modulates actin dynamics in motor neurons. Nat. Neurosci. 2016; 19(12):1610-1618.
42. Formoso K, Garcia M D, Frasch A C, Scorticati C. Evidence for a role of glycoprotein M6a in dendritic spine formation and synaptogenesis. Mol. Cell. Neurosci. 2016; 77:95-104.
43. Goyal S, Hamrah P. Understanding Neuropathic Corneal Pain—Gaps and Current Therapeutic Approaches. Semin Ophthalmol 2016; 31(1-2):59-70.
44. Zieglgansberger W. Substance P and pain chronicity. Cell Tissue Res. 2019; 375(1):227-241.
45. Ferrari, G. et al. Ocular Surface Injury Induces Inflammation in the Brain: In Vivo and Ex Vivo Evidence of a Corneal-Trigeminal Axis. Invest. Ophthalmol. Vis. Sci. 55, 6289-6300 (2014).
46. Parra A et al. Ocular surface wetness is regulated by TRPM8-dependent cold thermoreceptors of the cornea. Nat. Med. 2010; 16(12):1396-1399.
47. Proudfoot C J et al. Analgesia mediated by the TRPM8 cold receptor in chronic neuropathic pain. Curr. Biol. 2006; 16(16):1591-1605.
48. Liu B et al. TRPM8 is the Principal Mediator of Menthol-induced Analgesia of Acute and Inflammatory Pain. Pain 2013; 154(10):2169-2177.
49. Fernandez-Pella C, Viana F. Targeting TRPM8 for Pain Relief. The Open Pain Journal 2013; 6:154-164.
50. Hayashi M et al. Intrathecally administered Sema3A protein attenuates neuropathic pain behavior in rats with chronic constriction injury of the sciatic nerve. Neurosci. Res. 2011; 69(1):17-24.
51. Chen W et al. Rapamycin-Resistant mTOR Activity Is Required for Sensory Axon Regeneration Induced by a Conditioning Lesion [Internet]. eNeuro 2017; 3(6). doi:10.1523/ENEURO.0358-16.2016
52. Bligh E G, Dyer W J. A Rapid Method of Total Lipid Extraction and Purification. Can. J. Biochem. Physiol. 1959; 37(8):911-917.
53. Do, K. V. et al. Elovanoids counteract oligomeric β-amyloid-induced gene expression and protect photoreceptors. Proc. Natl. Acad. Sci. USA 116, 24317-24325 (2019).
54. Murphy P J, Lawrenson J G, Patel S, Marshall J. Reliability of the non-contact corneal aesthesiometer and its comparison with the Cochet-Bonnet aesthesiometer. Ophthalmic Physiol Opt 1998; 18(6):532-539.5
55. Belmonte C, Acosta M C, Schmelz M, Gallar J. Measurement of corneal sensitivity to mechanical and chemical stimulation with a CO2 esthesiometer. Invest. Ophthalmol. Vis. Sci. 1999; 40(2):513-519.
56. Picelli S et al. Full-length RNA-seq from single cells using Smart-seq2. Nat Protoc 2014; 9(1):171-181.
57. Liao Y, Smyth G K, Shi W. The R package Rsubread is easier, faster, cheaper and better for alignment and quantification of RNA sequencing reads. Nucleic Acids Res. 2019; 47(8):e47.
58. Liao Y, Smyth G K, Shi W. featureCounts: an efficient general purpose program for assigning sequence reads to genomic features. Bioinformatics 2014; 30(7):923-930.
59. Love M I, Huber W, Anders S. Moderated estimation of fold change and dispersion for RNA-seq data with DESeq2. Genome Biol. 2014; 15(12):550.
60. Kuleshov M V et al. Enrichr: a comprehensive gene set enrichment analysis web server 2016 update. Nucleic Acids Res. 2016; 44(W1):W90-97.

### Example 2

Discovery of a new RvD6 isomer that:
Promotes corneal wound healing, sensitivity and nerve regeneration.
Stimulates “beneficial” signaling back to trigeminal ganglia neurons.
Induces a genetic program in the trigeminal ganglia that repairs axon growth and decrease neuropathic pain.
This RvD6 isomer opens new therapeutic avenues for neurotrophic keratitis and dry eye-like pain.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating a corneal pathology in a subject in need thereof, the method comprising administering ocularly to the subject a composition comprising a therapeutically effective amount of:

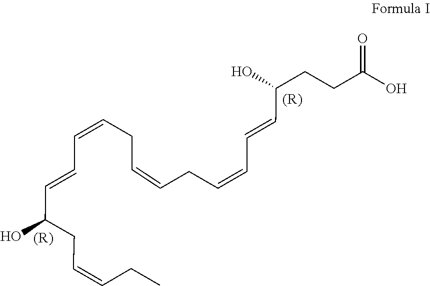

2. A method of protecting the cornea from a corneal pathology in a subject in need thereof, the method comprising administering ocularly to the subject a composition comprising a therapeutically effective amount of Formula I.

3. A method of promoting healing of a corneal pathology in a subject in need thereof, the method comprising administering ocularly to the subject a composition comprising a therapeutically effective amount of Formula I.

4. The method of claim 1, wherein treating a corneal pathology comprises increasing corneal nerve density, restoring corneal nerve density, repairing axon growth, inducing Rictor, inducing TIMP8 gene expression, wound healing, or a combination thereof.

5. The method of any one of claim 1, 2 or 3, wherein the corneal pathology comprises dry eye-disease (DED), photophobia, nerve damage, neuropathic pain, dry eye-like pain, corneal neurotrophic ulcers, trauma, a corneal wound, or neurotrophic keratitis.

6. The method of any one of claim 1, 2 or 3, wherein the composition further comprises a pharmaceutically acceptable carrier, excipient, or diluent.

7. The method of claim 6, wherein the pharmaceutically acceptable carrier, excipient, or diluent is suitable for topical administration.

8. The method of claim 1, 2 or 3 wherein the composition is formulated for topical administration.

9. The method of claim 6, wherein the pharmaceutical composition is formulated as an eye drop.

10. The method of any one of claim 1, 2 or 3, wherein the composition is administered hourly, daily, weekly, or monthly.

11. The method of claim 1, wherein a therapeutically effective amount comprises an amount between about 10 ng and about 1000 ng.
